SEQUENCE LISTING (39 sequences)

SEQ ID NO: 1 - protein, 429 aa, Mus musculus
MLAVVGAAALVLVAGAPWVLPSAAGGENLKPPENIDVYIIDDNYTLKWSSHGESMGSVTFSAEYRTKDEAKWLKVPECQHTTTTKCEFSLLDTNVYIKTQFRVRAEEGNSTSSWNEVDPFIPFYTAHMSPPEVRLEAEDKAILVHISPPGQDGNMWALEKPSFSYTIRIWQKSSSDKKTINSTYYVEKIPELLPETTYCLEVKAIHPSLKKHSNYSTVQCISTTVANKMPVPGNLQVDAQGKSYVLKWDYIASADVLFRAQWLPGYSKSSSGSRSDKWKPIPTCANVQTTHCVFSQDTVYTGTFFLHVQASEGNHTSFWSEEKFIDSQKHILPPPPVITVTAMSDTLLVYVNCQDSTCDGLNYEIIFWENTSNTKISMEKDGPEFTLKNLQPLTVYCVQARVLFRALLNKTSNFSEKLCEKTRPGSFST

SEQ ID NO: 2 - protein, 242 aa, Mus musculus
MRSRCTVSAVGLLSLCLVVSASLETITPSAFDGYPDEPCTINITIRNSRLILSWELENKSGPPANYTLWYTVMSKDENLTKVKNCSDTTKSSCDVTDKWLEGMESYVVAIVIVHRGDLTVCRCSDYIVPANAPLEPPEFEIVGFTDHINVTMEFPPVTSKIIQEKMKTTPFVIKEQIGDSVRKKHEPKVNNVTGNFTFVLRDLLPKTNYCVSLYFDDDPAIKSPLKCIVLQPGQESGLSESA

SEQ ID NO: 3 - protein, 436 aa, Homo sapiens
MMVVLLGATTLVLVAVAPWVLSAAAGGKNLKSPQKVEVDIIDDNFILRWNRSDESVGNVTFSFDYQKTGMDNWIKLSGCQNITSTKCNFSSLKLNVYEEIKLRIRAEKENTSSWYEVDSFTPFRKAQIGPPEVHLEAEDKAIVIHISPGTKDSVMWALDGLSFTYSLVIWKNSSGVEERIENIYSRHKIYKLSPETTYCLKVKAALLTSWKIGVYSPVHCIKTTVENELPPPENIEVSVQNQNYVLKWDYTYANMTFQVQWLHAFLKRNPGNHLYKWKQIPDCENVKTTQCVFPQNVFQKGIYLLRVQASDGNNTSFWSEEIKFDTEIQAFLLPPVFNIRSLSDSFHIYIGAPKQSGNTPVIQDYPLIYEIIFWENTSNAERKIIEKKTDVTVPNLKPLTVYCVKARAHTMDEKLNKSSVFSDAVCEKTKPGNTSK

SEQ ID NO: 4 - protein, 243 aa, Homo sapiens
MLLSQNAFIFRSLNLVLMVYISLVFGISYDSPDYTDESCTFKISLRNFRSILSWELKNHSIVPTHYTLLYTIMSKPEDLKVVKNCANTTRSFCDLTDEWRSTHEAYVTVLEGFSGNTTLFSCSHNFWLAIDMSFEPPEFEIVGFTNHINVMVKFPSIVEEELQFDLSLVIEEQSEGIVKKHKPEIKGNMSGNFTYIIDKLIPNTNYCVSVYLEHSDEQAVIKSPLKCTLLPPGQESESAESAK

SEQ ID NO: 5 - protein, 6 aa, Artificial Sequence
  Synthetic peptide
PVGLIG

SEQ ID NO: 6 - protein, 10 aa, Artificial Sequence
  Synthetic peptide
SGRSENIRTA

SEQ ID NO: 7 - protein, 166 aa, Homo sapiens
CDLPETHSLDNRRTLMLLAQMSRISPSSCLMDRHDFGFPQEEFDGNQFQKAPAISVLHELIQQIFNLFTTKDSSAAWDEDLLDKFCTELYQQLNDLEACVMQEERVGETPLMNADSILAVKKYFRRITLYLTEKKYSPCAWEVVRAEIMRSLSLSTNLQERLRRKE

SEQ ID NO: 8 - protein, 165 aa, Homo sapiens [sequence omitted]

SEQ ID NO: 9 - protein, 216 aa, Homo sapiens
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 10 - protein, 107 aa, Homo sapiens [sequence omitted]

SEQ ID NO: 11 - protein, 217 aa, Homo sapiens
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 12 - protein, 107 aa, Homo sapiens
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELK

SEQ ID NO: 13 - protein, 221 aa, Homo sapiens
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 14 - protein, 107 aa, Homo sapiens [sequence omitted]

SEQ ID NO: 15 - protein, 147 aa, Homo sapiens
FLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLV

SEQ ID NO: 16 - protein, 191 aa, Homo sapiens
MNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQENPCGPCSERRKHLFVQDPQTCKCSCKNTDSRCKARQLELNERTCRSLTRKD

SEQ ID NO: 17 - protein, 25 aa, Artificial Sequence
  Synthetic peptide
LLSQNAFIFRSLNLVLMVYISLVFG

SEQ ID NO: 18 - protein, 10 aa, Artificial Sequence
  Synthetic peptide
SGRSPAIFTA

SEQ ID NO: 19 - protein, 642 aa, Homo sapiens
ISYDSPDYTDESCTFKISLRNFRSILSWELKNHSIVPTHYTLLYTIMSKPEDLKVVKNCANTTRSFCDLTDEWRSTHEAYVTVLEGFSGNTTLFSCSHNFWLAIDMSFEPPEFEIVGFTNHINVMVKFPSIVEEELQFDLSLVIEEQSEGIVKKHKPEIKGNMSGNFTYIIDKLIPNTNYCVSVYLEHSDEQAVIKSPLKCTLLPPGQESESAESAKLEGGGGSSRSGRSPAIFTATGGGGGSGTCDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKERTGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 20 - protein, 565 aa, Homo sapiens
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVGGGGSDIQMTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKRTGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 21 - protein, 784 aa, Homo sapiens
MNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQENPCGPCSERRKHLFVQDPQTCKCSCKNTDSRCKARQLELNERTCRSLTRKDLEGGGGSSRSGRSPAIFTATGGGGGSGTEVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVGGGGSDIQMTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKRTGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 22 - protein, 560 aa, Homo sapiens
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVGGGGSDIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRTGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 23 - protein, 712 aa, Homo sapiens
FLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVGGGGSEVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVGGGGSDIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 24 - protein, 712 aa, Homo sapiens; identical to SEQ ID NO: 23

SEQ ID NO: 25 - protein, 397 aa, Homo sapiens
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKERTGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 26 - protein, 642 aa, Homo sapiens
ISYDSPDYTDESCTFKISLRNFRSILSWELKNHSIVPTHYTLLYTIMSKPEDLKVVKNCANTTRSFCDLTDEWRSTHEAYVTVLEGFSGNTTLFSCSHNFWLAIDMSFEPPEFEIVGFTNHINVMVKFPSIVEEELQFDLSLVIEEQSEGIVKKHKPEIKGNMSGNFTYIIDKLIPNTNYCVSVYLEHSDEQAVIKSPLKCTLLPPGQESESAESAKLEGGGGSSRSGRSPAIFTATGGGGGSGTCDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKERTGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 27 - protein, 735 aa, Homo sapiens
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVGGGGSDIQMTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKGGGGSCDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKERTGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 28 - protein, 949 aa, Homo sapiens
MNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQENPCGPCSERRKHLFVQDPQTCKCSCKNTDSRCKARQLELNERTCRSLTRKDLEGGGGSSRSGRSPAIFTATGGGGGSGTEVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVGGGGSDIQMTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKCDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKERTGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 29 - protein, 980 aa, Homo sapiens
ISYDSPDYTDESCTFKISLRNFRSILSWELKNHSIVPTHYTLLYTIMSKPEDLKVVKNCANTTRSFCDLTDEWRSTHEAYVTVLEGFSGNTTLFSCSHNFWLAIDMSFEPPEFEIVGFTNHINVMVKFPSIVEEELQFDLSLVIEEQSEGIVKKHKPEIKGNMSGNFTYIIDKLIPNTNYCVSVYLEHSDEQAVIKSPLKCTLLPPGQESESAESAKLEGGGGSSRSGRSPAIFTATGGGGGSGTEVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVGGGGSDIQMTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKGGGGSCDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKERTGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 30 - protein, 734 aa, Homo sapiens
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVGGGGSDIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKLEGGGGSGTCDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKERTGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 31 - protein, 882 aa, Homo sapiens
FLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVGGGGSEVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVGGGGSDIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKGGGGSCDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKERTGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 32 - protein, 975 aa, Homo sapiens
ISYDSPDYTDESCTFKISLRNFRSILSWELKNHSIVPTHYTLLYTIMSKPEDLKVVKNCANTTRSFCDLTDEWRSTHEAYVTVLEGFSGNTTLFSCSHNFWLAIDMSFEPPEFEIVGFTNHINVMVKFPSIVEEELQFDLSLVIEEQSEGIVKKHKPEIKGNMSGNFTYIIDKLIPNTNYCVSVYLEHSDEQAVIKSPLKCTLLPPGQESESAESAKLEGGGGSSRSGRSPAIFTATGGGGGSGTEVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVGGGGSDIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKGGGGSCDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKERTGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 33 - protein, 731 aa, Homo sapiens
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVGGGGSDILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKGGGGSCDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKERTGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 34 - protein, 976 aa, Homo sapiens
ISYDSPDYTDESCTFKISLRNFRSILSWELKNHSIVPTHYTLLYTIMSKPEDLKVVKNCANTTRSFCDLTDEWRSTHEAYVTVLEGFSGNTTLFSCSHNFWLAIDMSFEPPEFEIVGFTNHINVMVKFPSIVEEELQFDLSLVIEEQSEGIVKKHKPEIKGNMSGNFTYIIDKLIPNTNYCVSVYLEHSDEQAVIKSPLKCTLLPPGQESESAESAKLEGGGGSSRSGRSPAIFTATGGGGGSGTQVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVGGGGSDILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKGGGGSCDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKERTGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 35 - protein, 5 aa, Artificial Sequence
  Synthetic peptide
GGGGS

SEQ ID NO: 36 - protein, 5 aa, Artificial Sequence
  Synthetic peptide
GGGGS

SEQ ID NO: 37 - protein, 5 aa, Artificial Sequence
  Synthetic peptide
GGGGS

SEQ ID NO: 38 - protein, 397 aa, Homo sapiens
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKERTGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 39 - protein, 660 aa, Homo sapiens
MRSRCTVSAVGLLSLCLVVSASLETITPSAFDGYPDEPCTINITIRNSRLILSWELENKSGPPANYTLWYTVMSKDENLTKVKNCSDTTKSSCDVTDKWLEGMESYVVAIVIVHRGDLTVCRCSDYIVPANAPLEPPEFEIVGFTDHINVTMEFPPVTSKIIQEKMKTTPFVIKEQIGDSVRKKHEPKVNNVTGNFTFVLRDLLPKTNYCVSLYFDDDPAIKSPLKCIVLQPGQESGLSESAGGGGSSRSGRSPAIFTATGGGGGSGTCDLPHTYNLGNKRALTVLEEMRRLPPLSCLKDRKDFGFPLEKVDNQQIQKAQAILVLRDLTQQILNLFTSKDLSATWNATLLDSFCNDLHQQLNDLKACVMQEPPLTQEDSLLAVRTYFHRITVYLRKKKHSLCAWEVIRAEVWRALSSSTNLLARLSEEKEGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

(19) United States
(12) Patent Application Publication
(10) Pub. No.: US 2021/0252142 A1
(43) Pub. Date: Aug. 19, 2021

(54) COMPOSITIONS AND METHODS FOR TREATING VIRAL INFECTIONS

(71) Applicant: Aetio Biotherapy, Inc., Dallas, TX (US)

(72) Inventors: Yang Wang, Dallas, TX (US); Sherry Parker, Dallas, TX (US)

(21) Appl. No.: 17/168,554

(22) Filed: Feb. 5, 2021

Related U.S. Application Data
(60) Provisional application No. 62/970,774, filed on Feb. 6, 2020.

(51) Int. Cl. A61K 39/395; A61K 47/68; A61K 31/573; A61K 9/00; A61K 38/21; A61K 38/17; A61P 31/14; A61P 31/16

(52) U.S. Cl. CPC A61K 39/3955; A61K 47/6813; A61K 31/573; A61K 9/0073; A61P 31/16; A61K 38/215; A61K 38/217; A61K 38/1793; A61P 31/14; A61K 38/212

(57) ABSTRACT

The present invention includes composition and methods for treating a patient with a known or suspected viral-induced infection, respiratory disorder or exacerbation thereof, or preventing the same, the method comprising: administering to the patient in need thereof a therapeutically effective amount of an agent, wherein the agent comprises at least one of: (a) an activatable pro-IFN-Fc antibody, (b) an anti-viral-associated antibody or anti-epithelial-associated antibody (aVab/aEab)-IFN-Fc fusion protein, wherein the aVab/aEab is an anti-PD-L1, anti-VEGF, or anti-EGFR antibody variable domain, an activatable pro-aVab/aEab-IFN-Fc, an activatable aVab/aEab-pro-IFN-Fc, or pro-aVab/aEab-pro-IFN-Fc; wherein the fusion antibody prodrugs are activatable by proteases upregulated in upper and lower respiratory tracts during viral infection; wherein the preferred route of administration is via nasopharyngeal or oropharyngeal airways, and wherein the administration results in suppression of viral replication causing a reduction in the viral-induced infection, respiratory disorder or exacerbation thereof in the patient.

### CROSS-REFERENCE TO RELATED APPLICATIONS

This application claims priority to U.S. Provisional Application Ser. No. 62/970,774, filed Feb. 6, 2020, the entire contents of which are incorporated herein by reference.

### TECHNICAL FIELD OF THE INVENTION

The present invention relates in general to the field of treatments for viral infections.

### STATEMENT OF FEDERALLY FUNDED RESEARCH

Not applicable.

### INCORPORATION-BY-REFERENCE OF MATERIALS FILED ON COMPACT DISC

The present application includes a Sequence Listing which has been submitted in ASCII format via EFS-Web and is hereby incorporated by reference in its entirety. Said ASCII copy, created on Feb. 2, 2021, is named AEBI1002.txt and is 140,265 bytes in size.

### BACKGROUND OF THE INVENTION

Without limiting the scope of the invention, its background is described in connection with viral infections.

Currently there are few to no effective methods of treating respiratory viruses, including influenza and coronaviruses. The well-known SARS-CoV, MERS-CoV, and SARS-CoV2 have estimated mortality rates ranging between 2 and 35%. The only methods of prevention are isolation and containment. Viruses can rapidly replicate inside epithelial cells, infecting more cells in the respiratory tract, leading to local and even systemic damage by immune destruction and cytokine release syndrome. Suppression of viral replication is a way to reduce disease severity and it is known that type I interferon (IFN) can prevent the incidence or reduce severity of symptoms by way of reducing viral replication. However, recombinant IFN lacks targeting to viral entry points or sites of respiratory infection, has a short half-life, causes off-target side effects, and is subject to rapid degradation.

Recombinant IFN has generated significant interest as an acute anti-viral therapy. Many studies cite the impact of IFN deficiency in severity outcomes during the COVID pandemic. However, to this day there has not been an approved IFN for use in a respiratory illness due fear of exacerbating flu-like symptoms and cytokine storm at high doses and inconclusive efficacy in treatment and prevention at low doses. Also, historical concerns of using IFN as therapy stem from systemic and neurological side effects that caused patients suffering from hepatitis C infection to reduce dose to the point of inefficacy or to terminate treatment altogether. For example, Sleijfer, et al. Side effects of interferon-α therapy, Pharm World Sci (2005), summarizes the significant toxic side-effects of IFN therapy when trying to treat patients with Hepatitis C Virus (HCV), including: anemia, seizures, neuropathies, cardiomyopathy, retinal abnormalities, pneumonitis, and a wide variety of liver, connective tissue, dermatologic, hematologic, pulmonary, metabolic and neurologic disorders. This led to the recent publication of Wu and Metcalf, entitled “The Role of Type I IFNs in Influenza: Antiviral Superheroes or Immunopathogenic Villains?” J Innate Immun 2020; 12:437-447. The inability to develop a treatment against the influenza virus using IFN was found by Bennett, et al., Low-dose oral interferon alpha as prophylaxis against viral respiratory illness: a double-blind, parallel controlled trial during an influenza pandemic year, Influenza, Volume 7, Issue 5, Special Issue: Part 2 Pandemic H1N1 Papers, September 2013, Pages 854-862, which disappointingly found that, “Low dose oral IFNα prophylaxis did not reduce the incidence or impact of acute respiratory illness (ARI) or the impact of illness on daily activities.”

To reduce toxicity of IFN, a masked IFN pro-drug was developed against HCV, as taught in U.S. Pat. No. 10,523,549, issued to Stagliano, et al. This patent teaches the use of an IFN fusion protein that uses a peptide mask to block IFN activity. The peptide mask is a synthetically generated peptide with a sequence between 8-15 amino acids long that is cleaved by the HCV protease HCV-NS3/4, which is a nonstructural protein the hepatitis C virus produces intracellularly. This HCV-pro-IFN must enter the cell before the HCV protease cleaves the peptide mask to activate the IFN activity, and the method of use is limited to hepatitis C infection.

What is needed are novel agents that can safely treat a patient with a viral-induced infection, in particular respiratory disorders, or prevent exacerbation thereof.

### SUMMARY OF THE INVENTION

In one embodiment, the present invention includes a method of treating a patient with a viral infection, viral-induced infection, respiratory disorder or exacerbation thereof, the method comprising: administering to the patient in need thereof a therapeutically effective amount of an agent, wherein the agent comprises at least one of: a fusion protein comprising: (a) anti-viral-associated antibody or anti-epithelial-associated antibody-IFN-Fc (aVab/aEab-IFN-Fc), wherein the aVab/aEab is an anti-PD-L1, anti-VEGF, or anti-EGFR antibody variable domain); (b) an activatable pro-IFN-Fc fusion protein (pro-IFN-Fc); (c) a fusion protein comprising of an activatable pro-aVab/aEab-IFN-Fc, an activatable aVab/aEab-pro-IFN-Fc, or pro-aVab/aEab-pro-IFN-Fc; (d) an Fc-dimerized combination of aforementioned fusion proteins or (e) a polynucleotide that expresses the fusion proteins described in (a)-(d) in a target bronchial epithelial cell; and wherein the administration results in suppression of viral replication causing a reduction in the viral-induced infection, respiratory disorder or exacerbation thereof in the patient; and wherein the administration results in suppression of viral replication causing a reduction in the viral-induced infection, respiratory disorder or exacerbation thereof in the patient. In one aspect, the viral-induced exacerbation is caused by infection with a virus selected from the group consisting of Orthomyxoviridae, Paramyxoviridae, Picornaviridae, Rhabdoviridae, Coronaviridae, or Flaviviridae. In another aspect, the virus is seasonal influenza. In another aspect, the virus is SARS, SARS-CoV, MERS-CoV, 2019-nCoV virus or nCoV strains of subsequent years. In another aspect, the pro-IFN is activated by a protease that is upregulated or secreted during a viral infection. In another aspect, the protease is selected from membrane anchored MMPs, including MMP14 (MT1-MMP), MMP15 (MT2-MMP), MMP16 (MT3-MMP), MMP17 (MT4-MMP), MMP24 (MT5-MMP), MMP25 (MT5-MMP or leukolysin), or matrilysins MMP-7 and MMP-26, or stromelysins MMP3, MMP10, MMP11, MMP19, or gelatinases MMP2, MMP9, or collagenases MMP1, MMP8, MMP13, MMP18, or any one of caspase 1 to 9. In another aspect, the agent is formulated for administration by nasopharyngeal airway, oropharyngeal airway or intravenous delivery. In another aspect, the agent is administered to the lung airways with an aerosol nebulizer. In another aspect, the agent has at least 90, 91, 92, 93, 94, 95, 96, 97, 98, 99 or 100% sequence identify with a fusion protein as set forth in SEQ ID NO:19 to 34, 38 or 39. In another aspect, the administration of the agent is to nasopharyngeal airways. In another aspect, the administration of the agent is to the lower respiratory tract. In another aspect, the agent is administered simultaneously, separately or sequentially in combination with an additional therapeutic agent. In another aspect, the additional therapeutic agent is an inhaled corticosteroid. In another aspect, the agent comprises a polynucleotide vector that expresses in a target bronchial epithelial cell the polynucleotide that expresses an anti-Epidermal Growth Factor Receptor-interferon (anti-EGFR-IFN) fusion antibody, an anti-PD-L1-IFN fusion antibody, or a pro-IFN polypeptide. In another aspect, the IFN is selected from at least one of: IFN-α1, IFN-α2, IFN-α3, IFN-α4, IFN-α5, IFN-α6, IFN-α7, IFN-α8, IFN-α10, IFN-α13, IFN-α14, IFN-α16, IFN-α17, IFN-α21, IFN-β, IFN-ε, IFN-κ, IFN-ω. In another aspect, the pro-IFN is a pro-IFN-alpha, a pro-IFN-gamma, or a pro-IFN-lambda. In another aspect, the pro-IFN-alpha or pro-IFN-gamma further comprises an extracellular domain of IFNAR1 or IFNAR2. In another aspect, the pro-IFN-lambda further comprises an extracellular domain of IFNLR1. In another aspect, the pro-IFN is defined further as a heterodimer selected from at least one of: anti-VEGF(scFv)-Fc6-IFN-Fc9, anti-VEGF(scFv)-Fc6-pro-IFN-Fc9, pro-anti-VEGF(scFv)-Fc6-IFN-Fc9, pro-anti-VEGF(scFv)-Fc6-pro-IFN-Fc9, anti-EGFR(scFv)-Fc6-IFN-Fc9, anti-EGFR(scFv)-Fc6-pro-IFN-Fc9, anti-PD-L1(scFv)-Fc6-IFN-Fc9, anti-PD-L1(scFv)-Fc6-pro-IFN-Fc9, pro-anti-PD-L1(scFv)-Fc6-IFN-Fc9, or pro-anti-PD-L1(scFv)-Fc6-pro-IFN-Fc9. In another aspect, the pro-IFN is defined further as a homodimer selected from at least one of: pro-IFN-Fc, anti-VEGF(scFv)-IFN-Fc, anti-VEGF(scFv)-pro-IFN-Fc, pro-anti-VEGF(scFv)-IFN-Fc, anti-EGFR(scFv)-IFN-Fc, anti-EGFR(scFv)-pro-IFN-Fc, anti-PD-L1(scFv)-IFN-Fc, anti-PD-L1(scFv)-pro-IFN-Fc, pro-anti-PD-L1(scFv)-IFN-Fc, or pro-anti-PD-L1(scFv)-pro-IFN-Fc, IFN or pro-IFN fusion constructs can be fused to the N-terminus or C-terminus of Fc. In another aspect, the pro-IFN further comprises a peptide, a receptor to IFN, or a portion of IFN receptor that binds to and reduces the activity of IFN, and is disassociated with IFN-Fc. In another aspect, the pro-IFN further comprises an Fc region. In another aspect, the pro-IFN is activated in the bronchi by proteases secreted by cells infected with a virus.

In another embodiment, the present invention includes a method of administering to an individual prior to a viral infection, viral-induced infection, respiratory disorder or exacerbation thereof, the method comprising: administering to the individual a prophylactically effective amount of an agent, wherein the agent comprises at least one of: (a) aVab/aEab-IFN-Fc, wherein aVab/aEab=anti-viral-associated antibody or anti-epithelial-associated antibody (anti-PD-L1, anti-VEGF, or anti-EGFR); (b) an activatable pro-IFN-Fc fusion protein (pro-IFN-Fc); (c) a fusion protein comprising of an activatable pro-aVab/aEab-IFN-Fc, an activatable aVab/aEab-pro-IFN-Fc, or pro-aVab/aEab-pro-IFN-Fc; (d) any Fc-dimerized combination of aforementioned fusion proteins or (e) a polynucleotide that expresses the fusion proteins described in (a)-(d) in a target bronchial epithelial cell; and wherein the administration results in suppression of viral replication causing a reduction in the viral-induced infection, respiratory disorder or exacerbation thereof in the patient. In another aspect, the viral-induced exacerbation is caused by infection with a virus selected from the group consisting of Orthomyxoviridae, Paramyxoviridae, Picornaviridae, Rhabdoviridae, Coronaviridae, or Flaviviridae. In another aspect, the virus is SARS, SARs-CoV, MERS-CoV, or 2019-nCoV virus. In another aspect, the pro-IFN is activated by a protease that is upregulated or secreted during a viral infection. In another aspect, the protease is selected from membrane anchored MMPs MMP14 (MT1-MMP), MMP15 (MT2-MMP), MMP16 (MT3-MMP), MMP17 (MT4-MMP), MMP24 (MT5-MMP), MMP25 (MT5-MMP or leukolysin), or matrilysins MMP-7 and MMP-26, or stromelysins MMP3, MMP10, MMP11, MMP19, or gelatinases MMP2, MMP9, or collagenases MMP1, MMP8, MMP13, MMP18, or any one of caspase 1 to 9. In another aspect, the agent is formulated for administration by airway or intravenous delivery. In another aspect, the agent is administered to the lung airways with an aerosol nebulizer. In another aspect, the agent has at least 90, 91, 92, 93, 94, 95, 96, 97, 98, 99 or 100% sequence identify with a fusion protein as set forth in SEQ ID NO:19 to 34, 38 or 39. In another aspect, the administration of the agent is to the lower respiratory tract. In another aspect, the agent is administered simultaneously, separately or sequentially in combination with an additional therapeutic agent. In another aspect, the additional therapeutic agent is an inhaled corticosteroid. In another aspect, the agent comprises a polynucleotide vector that expresses in a target bronchial epithelial cell the polynucleotide that expresses an anti-Epidermal Growth Factor Receptor-interferon (anti-EGFR-IFN) fusion antibody, an anti-PD-L1-IFN fusion antibody, or a pro-IFN polypeptide. In another aspect, the IFN is selected from at least one of: IFN-α1, IFN-α2, IFN-α3, IFN-α4, IFN-α5, IFN-α6, IFN-α7, IFN-α8, IFN-α10, IFN-α13, IFN-α14, IFN-α16, IFN-α17, IFN-α21, IFN-β, IFN-ε, IFN-κ, IFN-ω. In another aspect, the pro-IFN is a pro-IFN-alpha, a pro-IFN-gamma, or a pro-IFN-lambda. In another aspect, the pro-IFN-alpha or pro-IFN-gamma further comprises an extracellular domain of IFNAR1 or IFNAR2. In another aspect, the pro-IFN-lambda further comprises an extracellular domain of IFNLR1. In another aspect, the pro-IFN is defined further as a heterodimer selected from at least one of: anti-VEGF(scFv)-Fc6-IFN-Fc9, anti-VEGF(scFv)-Fc6-pro-IFN-Fc9, pro-anti-VEGF(scFv)-Fc6-IFN-Fc9, pro-anti-VEGF(scFv)-Fc6-pro-IFN-Fc9, anti-EGFR(scFv)-Fc6-IFN-Fc9, anti-EGFR(scFv)-Fc6-pro-IFN-Fc9, anti-PD-L1(scFv)-Fc6-IFN-Fc9, anti-PD-L1(scFv)-Fc6-pro-IFN-Fc9, pro-anti-PD-L1(scFv)-Fc6-IFN-Fc9, or pro-anti-PD-L1(scFv)-Fc6-pro-IFN-Fc9. In another aspect, the pro-IFN is defined further as a homodimer selected from at least one of: pro-IFN-Fc, anti-VEGF(scFv)-IFN-Fc, anti-VEGF(scFv)-pro-IFN-Fc, pro-anti-VEGF(scFv)-IFN-Fc, anti-EGFR(scFv)-IFN-Fc, anti-EGFR(scFv)-pro-IFN-Fc, anti-PD-L1(scFv)-IFN-Fc, anti-PD-L1(scFv)-pro-IFN-Fc, pro-anti-PD-L1(scFv)-IFN-Fc, or pro-anti-PD-L1(scFv)-pro-IFN-Fc, IFN or pro-IFN fusion constructs can be fused to the N-terminus or C-terminus of Fc. In another aspect, the pro-IFN further comprise a peptide, a receptor to IFN, or a portion of IFN receptor that binds to and reduces the activity of IFN, and is disassociated with IFN-Fc. In another aspect, the pro-IFN further comprises an Fc region. In another aspect, the pro-IFN is activated in the bronchi by proteases secreted by cells infected with a virus.

In another embodiment, the present invention includes an agent that comprises at least one of: (a) aVab/aEab-IFN-Fc, wherein aVab/aEab=anti-viral-associated antibody or anti-epithelial-associated antibody (anti-PD-L1, anti-VEGF, or anti-EGFR); (b) an activatable pro-IFN-Fc fusion protein (pro-IFN-Fc); (c) a fusion protein comprising of an activatable pro-aVab/aEab-IFN-Fc, an activatable aVab/aEab-pro-IFN-Fc, or pro-aVab/aEab-pro-IFN-Fc; (d) any Fc-dimerized combination of the fusion proteins of (a)-(c); or (e) a polynucleotide that expresses the fusion proteins described in (a)-(d) in a target bronchial epithelial cell; wherein the agent is provided in an amount sufficient to suppress viral replication or reduction in viral burden. In one aspect, the agent is IFN (pro-IFN), X-pro-IFN or pro-IFN-X and is provided in an amount greater than 1 mg/kg per dose. In another aspect, the agent is IFN (pro-IFN), X-pro-IFN or pro-IFN-X and is provided in an amount of 1, 2, 3, 4, 5, 6, 7, 8, 9, 10, 15, 20, 25, 30, 35, 40, 45, 50, 55, 60 mg/kg per dose. In another aspect, the agent has at least 90, 91, 92, 93, 94, 95, 96, 97, 98, 99 or 100% sequence identify with a fusion protein as set forth in SEQ ID NO:19 to 34, 38 or 39.

In another embodiment, the present invention includes a polynucleotide that encodes at least one fusion protein selected from at least one of: (a) aVab/aEab-IFN-Fc, wherein aVab/aEab=anti-viral-associated antibody or anti-epithelial-associated antibody (anti-PD-L1, anti-VEGF, or anti-EGFR); (b) an activatable pro-IFN-Fc fusion protein (pro-IFN-Fc); (c) a fusion protein comprising of an activatable pro-aVab/aEab-IFN-Fc, an activatable aVab/aEab-pro-IFN-Fc, or pro-aVab/aEab-pro-IFN-Fc; (d) any Fc-dimerized combination of the fusion proteins of (a)-(c). A vector that comprises a polynucleotide that encodes at least one fusion protein selected from at least one of: (a) aVab/aEab-IFN-Fc, wherein aVab/aEab=anti-viral-associated antibody or anti-epithelial-associated antibody (anti-PD-L1, anti-VEGF, or anti-EGFR); (b) an activatable pro-IFN-Fc fusion protein (pro-IFN-Fc); (c) a fusion protein comprising of an activatable pro-aVab/aEab-IFN-Fc, an activatable aVab/aEab-pro-IFN-Fc, or pro-aVab/aEab-pro-IFN-Fc; (d) any Fc-dimerized combination of the fusion proteins of (a)-(c). A host cell that comprises a vector that comprises a polynucleotide that encodes at least one fusion protein selected from at least one of: (a) aVab/aEab-IFN-Fc, wherein aVab/aEab=anti-viral-associated antibody or anti-epithelial-associated antibody (anti-PD-L1, anti-VEGF, or anti-EGFR); (b) an activatable pro-IFN-Fc fusion protein (pro-IFN-Fc); (c) a fusion protein comprising of an activatable pro-aVab/aEab-IFN-Fc, an activatable aVab/aEab-pro-IFN-Fc, or pro-aVab/aEab-pro-IFN-Fc; (d) any Fc-dimerized combination of the fusion proteins of (a)-(c). In another aspect, the polynucleotide encodes a polypeptide having 90, 91, 92, 93, 94, 95, 96, 97, 98, 99 or 100% sequence identify with a fusion protein as set forth in SEQ ID NO:19 to 34, 38 or 39. A method of making a fusion protein comprising expressing a ribonucleic acid that encodes at least one fusion protein selected from at least one of: (a) aVab/aEab-IFN-Fc, wherein aVab/aEab=anti-viral-associated antibody or anti-epithelial-associated antibody (anti-PD-L1, anti-VEGF, or anti-EGFR); (b) an activatable pro-IFN-Fc fusion protein (pro-IFN-Fc); (c) a fusion protein comprising of an activatable pro-aVab/aEab-IFN-Fc, an activatable aVab/aEab-pro-IFN-Fc, or pro-aVab/aEab-pro-IFN-Fc; (d) any Fc-dimerized combination of the fusion proteins of (a)-(c) under conditions in which the fusion protein is translated. In another aspect, the fusion protein has at least 90, 91, 92, 93, 94, 95, 96, 97, 98, 99 or 100% sequence identify with a fusion protein as set forth in SEQ ID NO:19 to 34, 38 or 39.

### BRIEF DESCRIPTION OF THE DRAWINGS

For a more complete understanding of the features and advantages of the present invention, reference is now made to the detailed description of the invention along with the accompanying figures and in which:

FIG. 1 shows several different heterodimer constructs of the present invention, and in which, aVab/aEab=anti-viral-associated antibody or anti-epithelial-associated antibody including anti-PD-L1, anti-VEGF, or anti-EGFR, pSub=protease substrate (all MMPs and caspases), IFN=any of the type I, II and III interferons listed, PRO-c1=prodrug construct 1, refers to any strategy that prevents or reduces activity of the active drug, focuses on blocking the targeting antibody portion of the fusion protein, PRO-c2=prodrug construct 2, refers to any strategy that prevents or reduces activity of IFN, NC=not cleavable.

FIG. 2 shows several different homodimer constructs of the present invention, and in which, aVab=anti-viral antibody, aEab=anti-epithelial-target antibody (EGFR or PD-L1), pSub=protease substrate (all MMPs and caspases), IFN=any of the type I, II and III interferons listed, PRO-c1=prodrug construct 1, refers to any strategy that prevents or reduces activity of the active drug, focuses on blocking the targeting antibody portion of the fusion protein, PRO-c2=prodrug construct 2, refers to any strategy that prevents or reduces activity of IFN, NC=not cleavable.

FIG. 3 is a graph that shows that human Pro-IFN (SEQ ID NO:19) administered post-infection can suppress VSV infection after MMP14 cleavage.

FIG. 4 is a graph that shows that pre-treatment with human Pro-IFN (SEQ ID NO:19) reduces infection by VSV after MMP14 cleavage.

FIGS. 5A and 5B are Fluorescence Activated Cell Sorting (FACS) data from A549 in vitro culture infected with VSV-GFP.

FIGS. 6A to 6D are graphs that show VERO cell lines were pre-treated with IFN-Fc (SEQ ID NO: 38) or pro-IFN (SEQ ID NO:19), 2 hours later, cells were infected with VSV-G. Viral load was measured 2 days later. Relative viral infection levels of VSV-G were reported by RFP or luciferase. FIG. 6A—IFN-Fc (SEQ ID NO: 38), FIG. 6B—Pro-IFN (SEQ ID NO:19), FIG. 6C rIFN (R&D Systems, Cat #11100-1), and FIG. 6D—Pro-IFN digested.

FIGS. 7A to 7D are graphs that show ACE2 293 cells line were pre-treated with IFN-Fc (SEQ ID NO: 38) or pro-IFN (SEQ ID NO:19), 2 hours later, cells were infected with VSV-G. Viral load was measured 2 days later. Relative viral infection levels of VSV-G were reported by RFP or luciferase. FIG. 7A—IFN-Fc (SEQ ID NO: 38), FIG. 7B—Pro-IFN (SEQ ID NO:19), FIG. 7C rIFN (R&D Systems, Cat #11100-1), and FIG. 7D—Pro-IFN digested.

FIGS. 8A and 8B are graphs that show ACE2 293 cells line were pre-treated with IFN-Fc (SEQ ID NO: 38) or pro-IFN (SEQ ID NO:19), 2 hours later, cells were infected with SAR-CoV-2. Viral load was measured 2 days later. Relative viral infection levels of SARS-CoV-2 were reported by RFP or luciferase. FIG. 7A—IFN-Fc (SEQ ID NO: 38), FIG. 7B—Pro-IFN (SEQ ID NO:19), FIG. 7C rIFN (R&D Systems, Cat #11100-1), and FIG. 7D—Pro-IFN digested.

FIGS. 9A to 9E are graphs that show that Pro-IFN (SEQ ID NO:19) can suppress influenza-induced inflammation and infection in mice. FIG. 9A shows the expression of lung IL-6, FIG. 9B shows the expression of lung MCP-1, FIG. 9C shows the expression of H1N1 RNA normalized to GAPDH, FIG. 9D shows the expression of lung TNF, and FIG. 9E shows the expression of lung IFN-gamma.

FIGS. 10 to 25 shows maps of various constructs of the present invention, SEQ ID NOS:19 to 34, respectively.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not limit the invention, except as outlined in the claims.

As the COVID-19 pandemic swept across the globe, causing 1.6 million deaths as of December of 2020, the inventors recognized that a more rapid path for approval of therapies would be to repurpose existing agents that could alleviate infectious spread, mortality and to maximize existing assets to a development pipeline that meet the time constraints of a pandemic. The present inventors recognized that IFN-based MMP-activatable pro-drugs, already in the clinical development pipeline, could provide a promising option for controlling and preventing respiratory viral infection. Surprisingly, after the filing of the present specification, others recognized that certain proteases are upregulated during viral infections, such as influenza and SARS-CoV-2 infection. Ueland T, Holter J C, Holten A R, et al. Distinct and early increase in circulating MMP-9 in COVID-19 patients with respiratory failure. J Infect. 2020; 81(3):e41-e43. doi:10.1016/j.jinf2020.06.061. Talmi-Frank et al describes MT1-MMP as a prominent lung-tissue-remodeling protease that is active in influenza virus infection (Talmi-Frank D, Altboum Z, Solomonov I, et al. Extracellular Matrix Proteolysis by MT1-MMP Contributes to Influenza-Related Tissue Damage and Mortality. Cell Host Microbe. 2016; 20(4):458-470. doi:10.1016/j.chom.2016.09.005).

The present invention is directed to aerosol spray formulations having antiviral agents that cover viral entry points and sites of infection, reaching nasal passages and the lungs, that have a long half-life and are not readily degraded in the mucosa and lower respiratory tract. The present invention includes a variety of different fusion protein constructs that can be delivered to virally infected tissue, e.g., upper and lower respiratory tracts.

In one example, pro-IFNs for use in the methods of the present invention include those taught by Fu and Cao in US Publication No. 2020/0123227A1, relevant portions and sequences incorporated herein by reference. The pro-IFNs taught by Fu are said to be directed to interferon prodrugs and their use in treating cancer. These applicants find that one particular advantage to such constructs was their ability to exert powerful anti-tumor activity in vivo reduction in many of the significant toxicities associated with interferon therapy.

Examples of viral-induced exacerbation caused by viral infections include viruses selected from the group consisting of Orthomyxoviridae (such as influenza), Paramyxoviridae (such as respiratory syncytial virus), Picornaviridae (such as rhinoviruses), Coronaviridae, or Flaviviridae virus. The virus can be a SARS, SARs-CoV, MERS-CoV, 2019-nCoV virus, or a CoV of subsequent years.

As used herein, the terms “nucleic acid,” “polynucleotide,” and “oligonucleotide” are used interchangeably herein to refer to a polymer of at least three nucleotides. A nucleoside comprises a nitrogenous base linked to a sugar molecule. In a polynucleotide, phosphate groups covalently link adjacent nucleosides to form a polymer. The polymer can include natural nucleosides (e.g., adenosine, thymidine, guanosine, cytidine, uridine, deoxyadenosine, deoxythymidine, deoxyguanosine, and deoxycytidine), nucleoside analogs, chemically modified bases, biologically modified bases (e.g., methylated bases), intercalated bases, and/or modified sugars (e.g., modified purines or pyrimidines). See, Kornberg and Baker (1992) DNA Replication, 2nd Ed., Freeman, San Francisco, Calif.; Scheit (1980) Nucleotide Analogs, John Wiley, New York, N.Y.), and U.S. Patent Publication No. 20040092470 and references therein for further discussion of various nucleotides, nucleosides, and backbone structures that can be used in the polynucleotides described herein. A polynucleotide can have any length and sequence and can be single-stranded or double-stranded. Where this document provides a nucleic acid sequence, the complementary sequence also is provided. Further, where a sequence is provided as DNA, the corresponding RNA sequence (i.e., the sequence in which T is replaced by U) also is provided.

As used herein, the term “nucleic acid construct” refers to a nucleic acid that has been recombinantly modified or is derived from such a nucleic acid. For example, a nucleic acid construct can contain a mutation, deletion, or substitution relative to a naturally occurring nucleic acid molecule. A nucleic acid construct can comprise two or more nucleic acid segments that are derived from or originate from different sources such as different organisms (e.g., a recombinant polynucleotide). The sequence of one or more portions of a nucleic acid construct may be entirely invented by man.

As used herein, the “nucleic acid sequence” refers to the nucleic acid material itself and is not restricted to the sequence information (i.e., the succession of letters chosen among the five base letters A, G, C, T, or U) that biochemically characterizes a specific nucleic acid (e.g., DNA or RNA) molecule.

As used herein, the term “gene” has its meaning as understood in the art. In general, the term “gene” refers to a nucleic acid that includes a portion encoding a protein; the term optionally may encompass regulatory sequences such as promoters, enhancers, terminators, etc., in addition to coding sequences (open reading frames). This definition is not intended to exclude application of the term “gene” to non-protein coding expression units but rather to clarify that, in most cases, the term as used in this document refers to a protein-encoding nucleic acid. It will be appreciated that the definition of gene can include nucleic acids that do not encode proteins, but rather provide templates for transcription of functional RNA molecules such as tRNAs or rRNAs, for example.

As used herein, the terms “gene product” or “expression product” refer to, in general, an RNA transcribed from the gene or a polypeptide encoded by an RNA transcribed from the gene. Expression of a gene or a polynucleotide refers to (1) transcription of RNA from the gene or polynucleotide; (2) translation of RNA transcribed from the gene or polynucleotide, or both (1) and (2).

As used herein, the term “Vector” refers to a nucleic acid or a virus, viral genome, plasmid, or portion thereof that is a nucleic acid molecule that can replication and/or express a nucleic acid molecule in cell. Where the vector is a nucleic acid, the nucleic acid molecule to be transferred is generally linked to, e.g., inserted into, the vector nucleic acid molecule. A nucleic acid vector may include sequences that direct autonomous replication within suitable host cells (e.g., an origin of replication), or may include sequences sufficient to allow integration of part of all of the nucleic acid into host cell DNA. Useful nucleic acid vectors include, for example, DNA or RNA plasmids, cosmids, and naturally occurring or modified viral genomes or portions thereof, or nucleic acids (DNA or RNA) that can be packaged into viral capsids. Plasmid vectors typically include an origin of replication and one or more selectable markers. Plasmids may include part or all of a viral genome (e.g., a viral promoter, enhancer, processing or packaging signals, etc.). Viruses or portions thereof (e.g., viral capsids) that can be used to introduce nucleic acid molecules into cells are referred to as viral vectors. Useful animal viral vectors include adenoviruses, retroviruses, lentiviruses, vaccinia virus and other poxviruses, herpes simplex virus, and others. Useful plant viral vectors include those based on tobamoviruses, ilarviruses, etc. Viral vectors may or may not contain sufficient viral genetic information for production of infectious virus when introduced into host cells, i.e., viral vectors may be replication-defective, and such replication-defective viral vectors may be preferable for certain embodiments of the invention. Where sufficient information is lacking it may, but need not be, supplied by a host cell or by another vector introduced into the cell. An “expression vector” is a vector that includes one or more expression control sequences, and an “expression control sequence” is a DNA sequence that controls and regulates the transcription and/or translation of another DNA sequence.

As used herein, the term “operably linked” refers to a relationship between two nucleic acid sequences wherein the expression of one of the nucleic acid sequences is, e.g., controlled by, regulated by, or modulated by the other nucleic acid sequence. For example, transcription of a nucleic acid sequence is directed by an operably linked promoter sequence; post-transcriptional processing of a nucleic acid is directed by an operably linked processing sequence; translation of a nucleic acid sequence is directed by an operably linked translational regulatory sequence; transport or localization of a nucleic acid or polypeptide is directed by an operably linked transport or localization sequence; and post-translational processing of a polypeptide is directed by an operably linked processing sequence. A nucleic acid sequence that is operably linked to a second nucleic acid sequence typically is covalently linked, either directly or indirectly, to such a sequence, although any effective three-dimensional association is acceptable. It is noted that a single nucleic acid sequence can be operably linked to a plurality of other sequences. For example, a single promoter can direct transcription of multiple RNA species. A coding sequence is “operably linked” and “under the control” of an expression control sequence in a cell when RNA polymerase is able to transcribe the coding sequence into mRNA, which then can be translated into the protein encoded by the coding sequence.

Standard techniques for cloning, DNA isolation, amplification and purification, for enzymatic reactions involving DNA ligase, DNA polymerase, restriction endonucleases and the like, and various separation techniques are those known and commonly employed by those skilled in the art. A number of standard techniques are described in Sambrook et al. (1989) Molecular Cloning, Second Edition, Cold Spring Harbor Laboratory, Plainview, N.Y.; Maniatis et al. (1982) Molecular Cloning, Cold Spring Harbor Laboratory, Plainview, N.Y.; Wu (ed.) (1993) Meth. Enzymol. 218, Part I; Wu (ed.) (1979) Meth. Enzymol. 68; Wu et al. (eds.) (1983) Meth. Enzymol. 100 and 101; Grossman and Moldave (eds.) Meth. Enzymol. 65; Miller (ed.) (1972) Experiments in Molecular Genetics, Cold Spring Harbor Laboratory, Cold Spring Harbor, N.Y.; Old and Primrose (1981) Principles of Gene Manipulation, University of California Press, Berkeley; Schleif and Wensink (1982) Practical Methods in Molecular Biology; Glover (ed.) (1985) DNA Cloning Vol. I and II, IRL Press, Oxford, UK; Hames and Higgins (eds.) (1985) Nucleic Acid Hybridization, IRL Press, Oxford, UK; Setlow and Hollaender (1979) Genetic Engineering: Principles and Methods, Vols. 1-4, Plenum Press, New York; Fitchen, et al. (1993) Annu Rev. Microbiol. 47:739-764; Tolstoshev, et al. (1993) in Genomic Research in Molecular Medicine and Virology, Academic Press; and Ausubel et al. (1992) Current Protocols in Molecular Biology, Greene/Wiley, New York, N.Y. Abbreviations and nomenclature, where employed, are deemed standard in the field and commonly used in professional journals such as those cited herein. Many of the procedures useful for practicing the present invention, whether or not described herein in detail, are well known to those skilled in the arts of molecular biology, biochemistry, immunology, and medicine.

As used herein, the term “host cell” refers to cells into which a recombinant expression vector can be introduced. A host cell for use with the disclosed expression systems and methods typically is a eukaryotic cell, such as a plant cell. As used herein, “transformed” and “transfected” encompass the introduction of a nucleic acid molecule (e.g., a vector) into a cell by one of a number of techniques.

As used herein, the terms “polypeptide” refers to an amino acid chain, regardless of length or post-translational modification (e.g., glycosylation or phosphorylation). Polypeptides can include full length proteins or fragments or variants thereof. A “polypeptide of interest” refers to a target sequence expressed a cell, as described herein. In some embodiments, a polypeptide of interest can be a polypeptide that is not expressed in nature in the relevant type of cell or is not expressed at the level that the polypeptide is expressed when expression is achieved by intervention of the hand of man, as described herein. In certain embodiments, a polypeptide of interest can include sequences that are not naturally found in the relevant cell but are found naturally in other cell types or organisms.

As used herein, the term “fusion protein” refers to a hybrid protein, that includes portions of two or more different polypeptides, or fragments thereof, resulting from the expression of a polynucleotide that encodes at least a portion of each of the two polypeptides. Non-limiting examples of the fusion proteins of the present invention include: (a) an activatable pro-IFN-Fc antibody, (b) an aVab/aEab-IFN-Fc antibody, an activatable pro-aVab/aEab-IFN-Fc, an activatable aVab/aEab-pro-IFN-Fc, or pro-aVab/aEab-pro-IFN-Fc, wherein aVab/aEab is anti-VEGF, anti-EGFR or anti-PD-L1; wherein the fusion antibody prodrugs are activatable by proteases upregulated in upper and lower respiratory tracts during viral infection

As used herein, the term “identity” refers to the extent to which two or more nucleic acid sequences or two or more amino acid sequences are the same. The percent identity between two sequences over a window of evaluation is computed by aligning the sequences, determining the number of nucleotides or amino acids within the window of evaluation that are opposite an identical nucleotide or amino acid, allowing the introduction of gaps to maximize identity, dividing by the total number of nucleotides or amino acids in the window, and multiplying by 100.

Percent identity for any nucleic acid or amino acid sequence is determined as follows. First, a nucleic acid or amino acid sequence is compared to the identified nucleic acid or amino acid sequence using the BLAST 2 Sequences (B12seq) program from the stand-alone version of BLASTZ containing BLASTN version 2.0.14 and BLASTP version 2.0.14, or equivalents. This stand-alone version of BLASTZ can be accessed at the U.S. government's National Center for Biotechnology Information web site (World Wide Web at ncbi.nlm.nih.gov/blast/executables). Instructions explaining how to use the B12seq program can be found in the readme file accompanying BLASTZ.

In some cases, the nucleic acid or polypeptide sequence can include a sequence with at least 90 percent sequence identity (e.g., at least 90, 91, 92, 93, 94, 95, 96, 97, 98, or 99 percent sequence identity, or 100 percent sequence identity) to one or more sequences as set forth in SEQ ID NOS:20 to 34. In some embodiments, the fusion protein can have an amino acid sequence with at least 90 percent sequence identity (e.g., at least 90, 91, 92, 93, 94, 95, 96, 97, 98, or 99 percent sequence identity, or 100 percent sequence identity) to one or more sequences disclosed herein, however, certain specific sequences are those set forth in SEQ ID NOS:19 to 34, 38 or 39.

The pro-IFN is activated by a protease that is upregulated or secreted during a viral infection. Examples of protease include membrane anchored MMPs MMP14 (MT1-MMP), MMP15 (MT2-MMP), MMP16 (MT3-MMP), MMP17 (MT4-MMP), MMP24 (MT5-MMP), MMP25 (MT5-MMP or leukolysin), or matrilysins MMP-7 and MMP-26, or stromelysins MMP3, MMP10, MMP11, MMP19, or gelatinases MMP2, MMP9, or collagenases MMP1, MMP8, MMP13, MMP18, or any one of caspase 1 to 9.

Typically, the agent is formulated for administration by airway delivery, e.g., the agent is administered to the lung airways with an aerosol nebulizer, such that the agent is delivered to the upper and lower respiratory tracts. The agent may be administered simultaneously, separately or sequentially in combination with an additional therapeutic agent. An additional therapeutic agent can be an inhaled corticosteroid. In certain examples, the agent is a polynucleotide vector that expresses in a target bronchial epithelial cell the polynucleotide that expresses an anti-VEGF-IFN fusion antibody, anti-EGFR-IFN fusion antibody, an anti-PD-L1-IFN fusion antibody, a pro-IFN polypeptide, an anti-VEGF-pro-IFN fusion antibody, an anti-EGFR-pro-IFN fusion antibody, an anti-PD-L1-pro-IFN fusion protein, a pro-anti-VEGF-IFN fusion antibody, a pro-anti-PD-L1-IFN fusion antibody, a pro-anti-VEGF-pro-IFN fusion antibody, or a pro-anti-PD-L1-pro-IFN fusion antibody. The IFN can selected from at least one of: IFN-α1, IFN-α2, IFN-α3, IFN-α4, IFN-α5, IFN-α6, IFN-α7, IFN-α8, IFN-α10, IFN-α13, IFN-α14, IFN-α16, IFN-α17, IFN-α21, IFN-β, IFN-ε, IFN-κ, IFN-ω. The pro-IFN can be a pro-IFN-alpha, a pro-IFN-gamma, or a pro-IFN-lambda. If the agent is pro-IFN-alpha or pro-IFN-gamma, then it can further include an extracellular domain of IFNAR1 or IFNAR2. If the agent is pro-IFN-lambda it can further include an extracellular domain of IFNLR1.

The pro-IFN may be defined further as a heterodimer selected from at least one of: anti-VEGF(scFv)-Fc6-IFN-Fc9, anti-VEGF(scFv)-Fc6-pro-IFN-Fc9, pro-anti-VEGF(scFv)-Fc6-IFN-Fc9, pro-anti-VEGF(scFv)-Fc6-pro-IFN-Fc9, anti-EGFR(scFv)-Fc6-IFN-Fc9, anti-EGFR(scFv)-Fc6-pro-IFN-Fc9, anti-PD-L1(scFv)-Fc6-IFN-Fc9, anti-PD-L1(scFv)-Fc6-pro-IFN-Fc9, pro-anti-PD-L1(scFv)-Fc6-IFN-Fc9, or pro-anti-PD-L1(scFv)-Fc6-pro-IFN-Fc9. The pro-IFN can further include a peptide, a receptor to IFN, or a portion of IFN receptor that binds to and reduces the activity of IFN, and is disassociated with IFN-Fc. The homodimer construct may have anti-EGFR or anti-PD-L1 linked to the N′-terminal of Fc, while the IFN or pro-IFN can be linked to the C′-terminal of Fc. The pro-IFN is activated in the bronchi by proteases secreted by cells infected with a virus.

Prior to the present invention, it has been shown that systemic or intratumoral delivery of pro-IFN can be used to reduce tumor burden and increase immunity against cancer (Fu and Cao, US 2020/0123227A1). Anti-EGFR-IFNβ was also previously shown to have anti-tumor activity in Yang X, Zhang X, Fu M L, et al. Targeting the tumor microenvironment with interferon-β bridges innate and adaptive immune responses. Cancer Cell. 2014; 25(1):37-48. doi:10.1016/j.ccr.2013.12.00. Low dose anti-EGFR aerosol therapy was tested by De Santis, et al for the treatment of lung metastases in animal models and have demonstrated an ability to reduce metastasis. De Santis R, Rosi A, Anastasi A M, et al. Efficacy of aerosol therapy of lung cancer correlates with EGFR paralysis induced by AvidinOX-anchored biotinylated Cetuximab. Oncotarget. 2014; 5(19):9239-9255. doi:10.18632/oncotarget.2409.

Clinical trials assessing therapeutic and preventative ability of interferon-alpha-2 for the common cold, influenza and other respiratory infections have had mixed results. Hayden et al (J Infect Dis. 1984) assessed therapeutic efficacy in two randomized, double-blind studies treating volunteers with intranasal spray or drops 28 hours after rhinovirus inoculation but did not find statistically significant prevention of infection or colds in either study (Hayden F G, Gwaltney J M Jr. Intranasal interferon-alpha 2 treatment of experimental rhinoviral colds. J Infect Dis. 1984 August; 150(2):174-80. doi: 10.1093/infdis/150.2.174. PMID: 6381610). Gao et al's study of military recruits was suggestive that low dose rIFN α-2b could be used to prevent infections caused by influenza A virus, influenza B virus, and parainfluenza viruses 1-3 but not RSV (Gao L, Yu S, Chen Q, et al. A randomized controlled trial of low-dose recombinant human interferons alpha-2b nasal spray to prevent acute viral respiratory infections in military recruits. Vaccine. 2010; 28(28):4445-4451. doi:10.1016/j.vaccine.2010.03.062). rhIFN-α nasal drops were tested in a non-randomized prospective study to prevent coronavirus disease in medical staff, though inconclusive, is suggestive of potential and warranting further study (Meng, Zhongji & Wang, Tongyu & Li, Chen & Chen, Xinhe & Li, Longti & Qin, Xueqin & Li, Hai & Luo, Jie. 2020. An experimental trial of recombinant human interferon alpha nasal drops to prevent coronavirus disease 2019 in medical staff in an epidemic area. 10.1101/2020.04.11.20061473). Bennett, at al., disappointingly found that, “[l]ow dose oral IFNα prophylaxis did not reduce the incidence or impact of acute respiratory illness (ARI) or the impact of illness on daily activities”. However, the study employed lozenge delivery methods which the present inventors believe is insufficient to target influenza in proper sites of viral entry and infection such as nasal passages and lower respiratory tract.

The present invention uses high doses of pro-IFN-Fc, anti-EGFR-IFN-Fc, pro-anti-EGFR-IFN-Fc, anti-PD-L1-IFN-Fc fusion antibodies to treat respiratory infections. As used herein, low doses refer to dosing of less than 0.1 mg/kg body weight of the subject. By contrast, the high dosing regimen of the present invention can include 1, 2, 3, 4, 5, 6, 7, 8, 9, 10, 15, 20, 25, 30, 35, 40, 45, 50, 55, 60 mg/kg per dose. These doses can be given tid, bid, or even once daily as a result of the significantly longer half-life of the construct herein. For example, recombinant IFN (rIFN) has a half-life of about 3 hours, while the pro-IFN-Fc has a half-life of 90-95 hours. Thus, the dosing herein is at least 10×, 20×, 30×, 40×, 50×, or 60× the normal dose for rIFN, without side effects. Pro-IFN-Fc has a dosing specificity that can be 1000× that of rIFN due to its superior safety profile.

Thus, the fusion proteins of the present invention include the use of an aerosol of antibody-based biologics or pro-cytokines for viral infections. For improved targeting and retention to the lungs, anti-viral-associated or anti-epithelial-associated antibodies such as anti-VEGF, anti-PD-L1 and anti-EGFR were also fused to IFN and pro-IFN. Lungs are the predominant source of VEGF in the lungs. Ectodomain-based prodrug antibody fusions were designed to prevent toxicity as well. VEGF, which promote angiogenesis but also induce vascular leakiness and permeability, were observed to be higher in ICU and non-ICU COVID-19 patients than healthy controls. Thus, fusion of anti-VEGF to IFN is designed to confer a secondary effect of curbing vessel-permeabilization leading to ARDS. Further, an aerosol of anti-EGFR-IFN-Fc can increase its target to the epithelial cells to prevent and reduce infection since EGFR is highly expressed on those cells and antibody can retain IFN longer on lung epithelial cells. Virally infected cells express IFN which can rapidly induce PD-L1 expression of epithelial cells. Therefore, anti-PD-L1-IFN can target those infected cells more effectively.

Finally, virally infected tissues have higher levels of MMP that help to perpetrate viral invasion. As MT1-MMP has been implicated in tissue damage associated with influenza viral infection, Pro-IFN and the other MMP-activatable proteins of the present invention can compete to interact with MMPs, become activated, and reduce the viral load in infected cells, as well as reduce inflammation. MMPs are key proteases increasing viral and immune cells invading lung leading to clinical features and pathology.

Due to the stable nature of antibody-based biologics, the fusion proteins of the present invention can be stored for longer durations than recombinant IFN. Stability is conferred by capping of the IFN and/or antibody binding sites with endogenous ectodomains in addition to the stability of IgG globular proteins due their optimal folding structure and disulfide bonds. The improved stability, in addition to the attenuation of off-target toxicities, allows for practical and meaningful use of IFN in diverse situations. For example, users can carry pro-IFN-Fc or other fusion proteins of the present invention to places without refrigeration for weeks and use it to prevent or treat infection during early disease phases. This would not be possible with rIFN, which requires refrigeration and may only be stable for less than 2 weeks. Furthermore, storage of rIFN in high concentrations is necessary to protect the cytokine from loss of bioactivity, which requires users to dilute and prepare the sample before administration as one cannot receive high doses of rIFN without the risk of side effects. Due to neonatal Fc receptor (FcRn) expressed by epithelial, endothelial and myeloid lineages in humans, IgG antibody-based IFN fusion proteins have increased in vivo half-life and decreased degradation in mucosa compared to rIFN, allowing for more infrequent dosing and conferring longer protection for the user.

The stability may also enable use of the drug and/or prophylactic in developing countries or rural areas where access to regular refrigeration is less likely. Military servicemen living in crowded environments and on active duty can benefit, as well as civilians entering public spaces such as airplanes or conferences during a pandemic. Hospitals can maintain an inventory of such drugs to protect against an outbreak and to protect medical staff prior to entry of high risk scenarios, e.g. intubating patients in the ICU during a respiratory viral outbreak. The present invention can be formulated into aerosols.

For delivery to the nasal or bronchial membranes, typically an aerosol formulation will be employed. The term “aerosol” includes any gas-borne suspended phase of the pharmacological agent which is capable of being inhaled into the bronchioles or nasal passages. Specifically, aerosol includes a gas-borne suspension of droplets of the compounds of the instant invention, as may be produced in a metered dose inhaler or nebulizer, or in a mist sprayer. Aerosol also includes a dry powder composition of a compound of the pharmacological agent suspended in air or other carrier gas, which may be delivered by insufflation from an inhaler device, for example.

For solutions used in making aerosols, the preferred range of concentration of the fusion proteins herein are from, e.g., 0.1-100 milligrams (mg)/milliliter (mL), more preferably 0.1-30 mg/mL, and most preferably, 1-10 mg/mL. Usually the solutions are buffered with a physiologically compatible buffer such as phosphate or bicarbonate. The usual pH range is 5 to 9, preferably 6.5 to 7.8, and more preferably 7.0 to 7.6. Typically, sodium chloride is added to adjust the osmolarity to the physiological range, preferably within 10% of isotonic. Formulation of such solutions for creating aerosol inhalants is discussed in Remington's Pharmaceutical Sciences, see also, Ganderton and Jones, DRUG DELIVERY TO THE RESPIRATORY TRACT, Ellis Horwood (1987); Gonda (1990) Critical Reviews in Therapeutic Drug Carrier Systems 6:273-313; and Raeburn et al. (1992) J. Pharmacol. Toxicol. Methods 27:143-159.

Solutions of the fusion proteins herein may be converted into aerosols by any of the known means routinely used for making aerosol inhalant pharmaceutical. In general, such methods comprise pressurizing or providing a means of pressurizing a container of the solution, usually with an inert carrier gas, and passing the pressurized gas through a small orifice, thereby pulling droplets of the solution into the mouth and trachea of the animal to which the drug is to be administered. Typically, a mouthpiece is fitted to the outlet of the orifice to facilitate delivery into the mouth and trachea.

For reasons of patient compliance, the methods for regulating the rate of lamellar body (or membrane coating granule) extrusion in mucosal membranes will typically be accomplished via pharmacological intervention. Transmucosal (i.e., sublingual, rectal, colonic, pulmonary, buccal and vaginal) drug delivery provides for an efficient entry of active substances to systemic circulation and reduce immediate metabolism by the liver and intestinal wall flora (See Chien Y. W., NOVEL DRUG DELIVERY SYSTEMS, Chapter 4 “Mucosal Drug Delivery,” Marcel Dekker, Inc. (1992). Transmucosal drug dosage forms (e.g., tablet, suppository, ointment, gel, pessary, membrane, and powder) are typically held in contact with the mucosal membrane and disintegrate and/or dissolve rapidly to allow immediate local and systemic absorption.

The fusion proteins of the present invention can also be developed as an oral formulation for cases in which digestive viral delivery has been observed. Oral delivery can include as a liquid or a solid, e.g., a lozenge, tablet, or capsule. The method of manufacture of these formulations are known in the art, including but not limited to, the addition of the pharmacological agent to a pre-manufactured tablet; cold compression of an inert filler, a binder, and either a pharmacological agent or a substance containing the agent (as described in U.S. Pat. No. 4,806,356); and encapsulation.

The present invention allows for a high dose of IFN-based therapy that avoids local and systemic toxicity and overcomes the problems of current low dose cancer therapies.

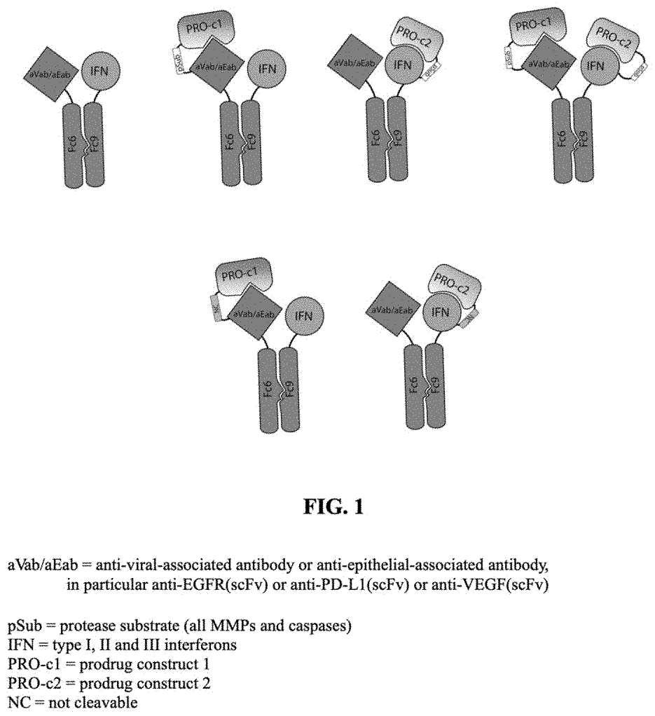

FIG. 1 shows several different constructs of the present invention, and in which, aVab=anti-viral antibody, aEab=anti-epithelial-target antibody (EGFR or PD-L1), pSub=protease substrate (all MMPs and caspases), IFN=any of the type I, II and III interferons listed, PRO-c1=prodrug construct 1, refers to any strategy that prevents or reduces activity of the active drug, focuses on blocking the targeting antibody portion of the fusion protein, PRO-c2=prodrug construct 2, refers to any strategy that prevents or reduces activity of the active drug, focuses on blocking IFN, NC=not cleavable.

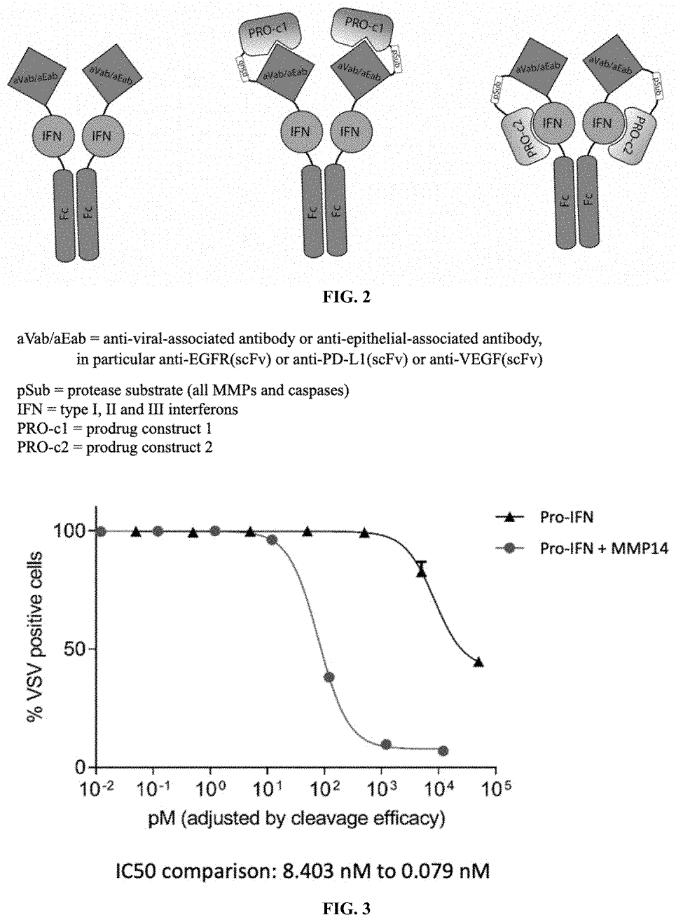

FIG. 2 shows several different homodimer constructs of the present invention, and in which aVab/aEab=anti-viral-associated antibody or anti-epithelial-associated antibody including anti-PD-L1, anti-VEGF, or anti-EGFR, pSub=protease substrate (all MMPs and caspases), IFN=any of the type I, II and III interferons listed, PRO-c1=prodrug construct 1, refers to any strategy that prevents or reduces activity of the active drug, focuses on blocking the targeting antibody portion of the fusion protein, PRO-c2=prodrug construct 2, refers to any strategy that prevents or reduces activity of the active drug, focuses on blocking IFN, NC=not cleavable.

Furthermore, the specificity of the present invention can be increased by selecting activating peptides that are susceptible to matrix metalloproteinases (MMPs) in tissues open for viral invasion. Inflamed or infected cells express higher levels of MMP. Thus, the present invention includes pro-formulations of MMP-sensitive anti-EGFR-IFNs to target MMP enriched tissues to help reduce side-effects and off-target toxicities. The pro-antibodies can also compete with viruses for MMP action, which contributes to tissue damage during viral infections such as influenza. A low dose of recombinant IFN may not be effective (due to toxicity and short half-life), while the pro-drug of the present invention allows for a greater therapeutic index and use of the therapy in higher doses.

Viral inhibition assays were conducted to demonstrate prophylactic capability and impact of post-infection therapy by pre-treating before infection or treating post-infection, respectively, across several cell lines, viruses and mouse models.

Inhibition of A549/293/VERO+H1N1/VSV/SARS-CoV2. MMP-14 Activation and Digestion. MMP-14 (MT1-MMP) and Recombinant Human Furin Protein were acquired from R&D Systems. MMP-14 was activated in a mixture of rhFurin and activation buffer, which was a pH 9.0 buffer comprised of 50 mM Tris, 1 mM CaCl₂, 0.5% Brij-35 and passed through a 0.2 μm filter. Final concentration of MMP-14 and rhFurin was 50 ng/μl and 0.86 ng/μl, respectively. MMP-14 and pro-IFN were co-incubated in assay buffer comprised of 50 mM Tris, 3 mM CaCl₂, 1 μM ZnCl2 and passed through 0.2 μm filter at a 1:5 mole ratio at 37° C. overnight.

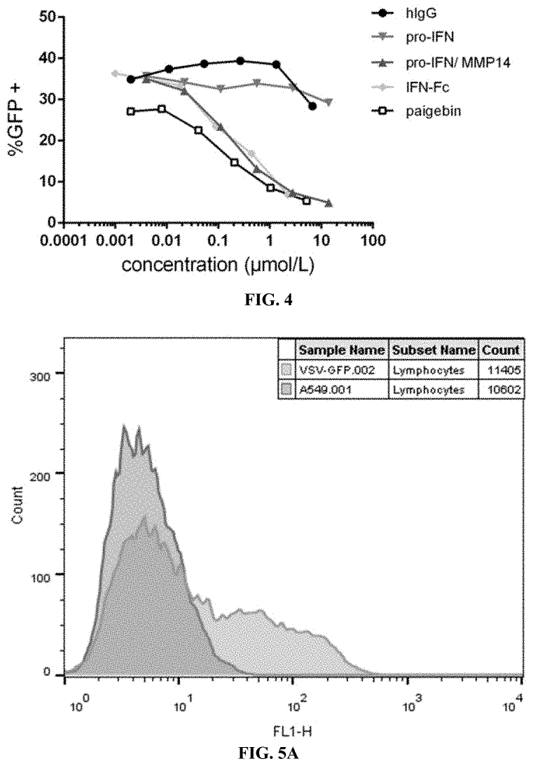

FIG. 3 is a graph that shows that Pro-IFN (SEQ ID NO:19) administered post-infection can suppress VSV infection after MMP-14 cleavage.

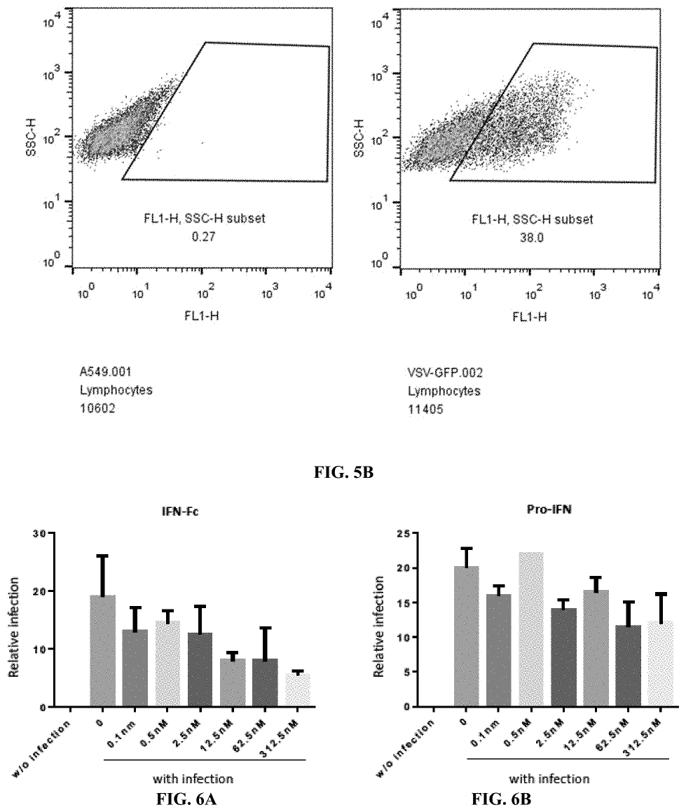

FIG. 4 is a graph that shows that Pre-treatment with human Pro-IFN (SEQ ID NO:19) reduces infection by VSV after MMP14 cleavage.

VSV-GFP Viral Inhibition Assays. 6×10⁴ A549 cells/well were plated at 600 ul/well in 10% FBS 1640 medium, 5% CO₂ at 37° C. overnight. Medium was aspirated. VSV-GFP was added at MOI=5 in 2% FBS of 1640 medium diluent and incubated at 37° C., 5% CO₂. After a 2-hour incubation period, serial dilutions of digested and non-digested pro-IFN were added and incubated at 37° C., 5% CO₂. 30-hours post-infection, cells were collected and fixed with 4% PFA for 20 minutes. GFP measurements were acquired and analyzed using LSF Fortessa Flow Cytometer (BD Biosciences) and FlowJo, respectively. GFP+ were defined as virally infected cells.

Next, 6×10⁴ A549 cells/well were plated at 600 ul/well in 10% FBS 1640 medium, 5% CO₂ at 37° C. overnight. Medium was aspirated. Each test and control sample (digested pro-IFN, non-digested pro-IFN (SEQ ID NO:19) and human IgG) was prepared with 5-fold serial dilutions using 10% FBS 1640 medium as diluent, starting at 1000 ng/ml. For IFN-Fc (SEQ ID NO:38) and paigebin, 5-fold serial dilutions were also prepared starting from 100 ng/ml. Final volume of each sample incubated with A549 cells was 200 μl.

Samples were incubated at 37° C. for 8-12 hours. Medium was aspirated. VSV-GFP was added at MOI=5 in 2% FBS of 1640 medium diluent and incubated at 37° C., 5% CO₂ for 16-24 hours. Cells were collected and fixed with 4% PFA for 20 minutes. GFP measurements were acquired and analyzed using LSF Fortessa Flow Cytometer (BD Biosciences) and FlowJo, respectively. GFP+ were defined as virally infected cells.

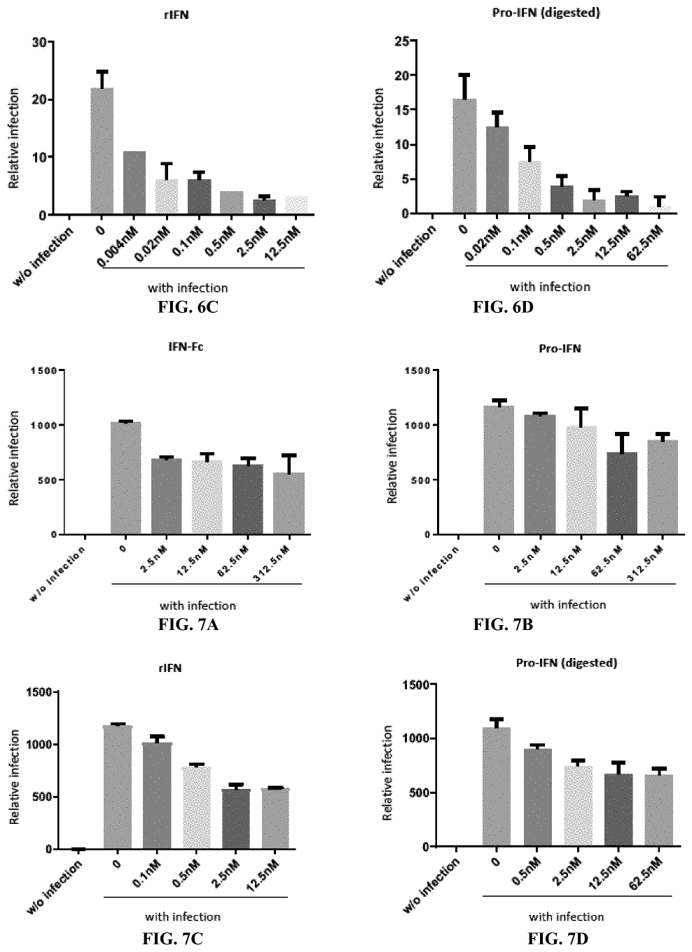

FIGS. 5A and 5B are Fluorescence Activated Cell Sorting (FACS) data from A549 in vitro culture infected with VSV-GFP.

Generating Pseudotyped Virus Cell Lines. Lenti-X™ 293 cells were infected with ACE2-expressing lentivirus to establish ACE2 expressing cell lines. Lenti-X™ 293 was purchased from Clontech/Takara Bio. Plasmids encoding SARS-CoV-2 Spike and ACE2 were obtained from MolecularCloud (Nanjing, China) and sub-cloned to a pCDH-EF lentiviral expression vector (System Biosciences) containing a puromycin resistance marker.

After selection with puromycin, pooled resistant cells were identified by flow cytometry analysis. Cell culture media was supplemented with 10% heat-inactivated fetal bovine serum, 2 mmol/L L-glutamine, 100 units/mL penicillin, and 100 μg/mL streptomycin. Lenti-X™ 293, VERO and their derivatives were cultured with complete DMEM medium (Sigma-Aldrich).

Lenti-X™ 293 cells were transfected with lentivirus package component plasmids, Gap/pol (Addgene #12251), RSV-Rev (Addgene #12253), pCDH-EF-IRFP-luc and pcDNA3.1(+)-2019-nCoV-spike-P2A-eGFP (MolecularCloud, #MC_0101087). Supernatants containing SARS-CoV-2 pseudotyped viral particles were collected 48- and 72-hours post-transfection for direct usage. Viral titer in TU/mL was determined by flow cytometry analysis of transduced Lenti-X™ 293-ACE2 cells. Similarly, pcDNA3.1(+)-2019-nCoV-spike-P2A-eGFP was replaced with pCMV-VSV-G (Addgene #8454) to generate the VSV-pseudotyped virus.

Pseudotyped Viral Inhibition Assay. 2.5×10⁴ cells/well of 293-ACE2 or 1.0×10⁴ cells/well of VERO cells were seeded in 96-well plates. 1-day later, cells were incubated with the indicated concentration of IFN-Fc (SEQ ID NO:38), Pro-IFN (SEQ ID NO:19), and digested Pro-IFN. SARS-CoV-2 or VSV pseudotyped lentiviral particles were inoculated on 293-ACE2 monolayers in the presence of 10 μg/ml of polybrene (Sigma-Aldrich) at the MOI of 0.01 or 0.5, respectively, and further incubated at 37° C. for 48-hours. VSV pseudotyped lentiviral particles were inoculated on VERO monolayers in the presence of 10 μg/ml of polybrene (Sigma-Aldrich) at a MOI of 0.75, and further incubated at 37° C. for 48-hours. IRFP reporter activity was measured on a CytoFLEX S Flow Cytometer (Beckman Coulter). The relative infection was calculated as the IRFP⁺ cell post-infection.

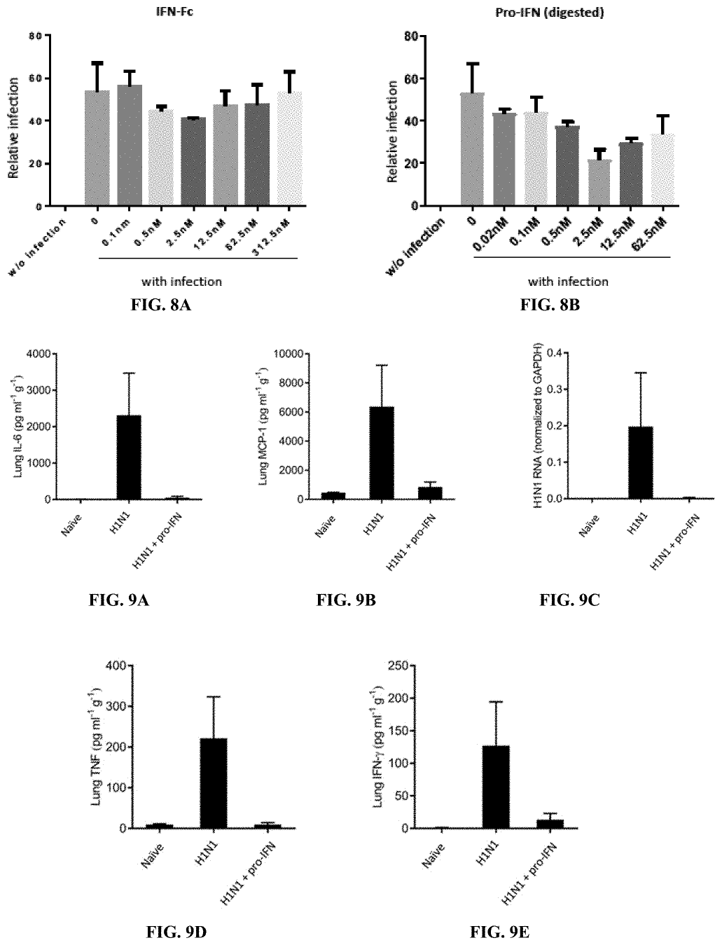

FIGS. 6A to 6D are graphs that show VERO cell lines were pre-treated with IFN-Fc (SEQ ID NO:38) or pro-IFN (SEQ ID NO:19), 2 hours later, cells were infected with VSV-G. Viral load was measured 2 days later. Relative viral infection levels of VSV-G were reported by RFP or luciferase. FIG. 6A—IFN-Fc (SEQ ID NO:38), FIG. 6B—Pro-IFN (SEQ ID NO:19), FIG. 6C rIFN (R&D Systems, Cat #11100-1), and FIG. 6D—Pro-IFN digested (SEQ ID NO:19).

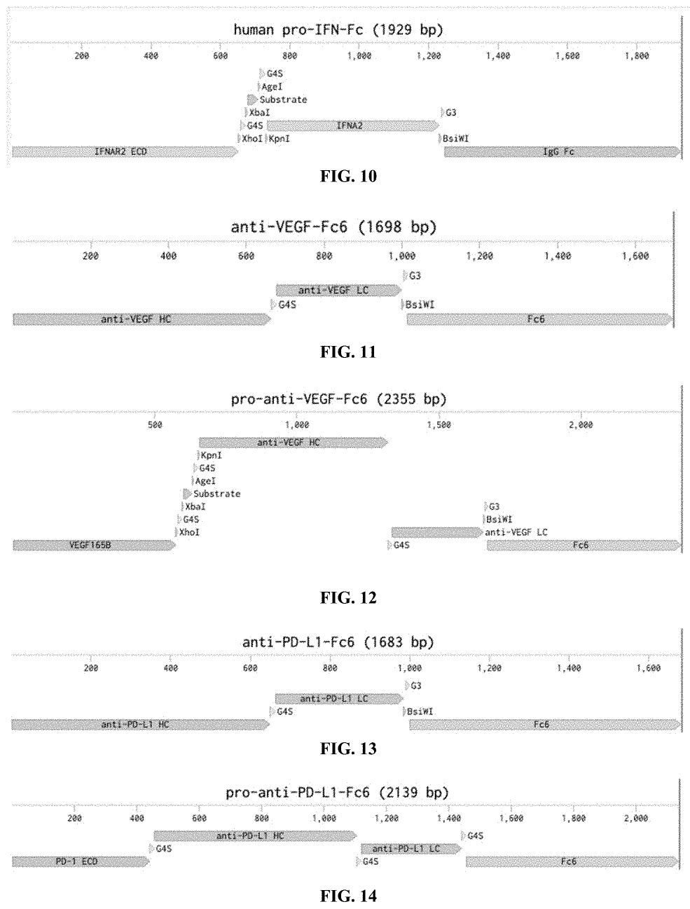

FIGS. 7A to 7D are graphs that show ACE2 293 cells line were pre-treated with IFN-Fc (SEQ ID NO:38) or pro-IFN (SEQ ID NO:19), 2 hours later, cells were infected with VSV-G. Viral load was measured 2 days later. Relative viral infection levels of VSV-G were reported by RFP or luciferase. FIG. 7A—IFN-Fc (SEQ ID NO:38), FIG. 7B—Pro-IFN (SEQ ID NO:19), FIG. 7C rIFN (R&D Systems, Cat #11100-1), and FIG. 7D—Pro-IFN digested (SEQ ID NO:19).

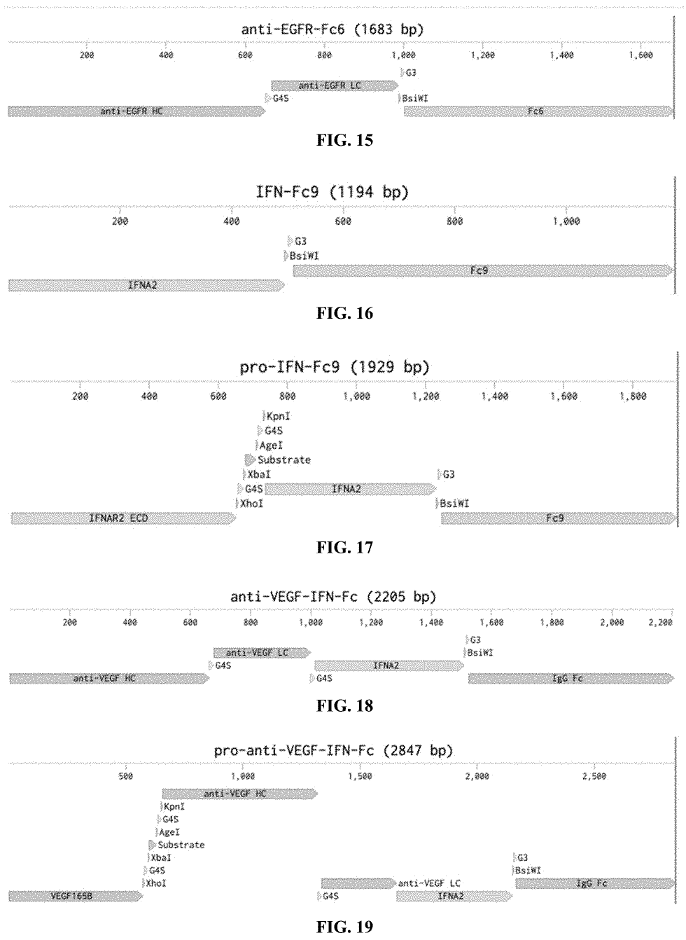

FIGS. 8A and 8B are graphs that show ACE2 293 cells line were pre-treated with IFN-Fc (SEQ ID NO:38) or pro-IFN (SEQ ID NO:19), 2 hours later, cells were infected with SAR-CoV-2. Viral load was measured 2 days later. Relative viral infection levels of SARS-CoV-2 were reported by RFP or luciferase. FIG. 7A—IFN-Fc (SEQ ID NO:38), FIG. 7B—Pro-IFN (SEQ ID NO:19), FIG. 7C rIFN (R&D Systems, Cat #11100-1), and FIG. 7D—Pro-IFN digested (SEQ ID NO:19).

In vivo—Mouse Construct Data. C57BL/6 mice were intra-nasally infected with influenza A/PR/8/34 (H1N1) virus at 50000 EID50/mouse. Pro-IFN (SEQ ID NO:39) was intra-nasally delivered at a dose of 25 ug/mouse at 5 hours post-infection. Naïve (mock) and H1NA infection alone served as negative and positive controls (n=4 per group). Lung tissue inflammatory cytokines and H1N1 virus RNA were quantified by BD™ CBA mouse inflammation kit (BD Biosciences) and RT-PCR. Data are expressed as mean SEM.

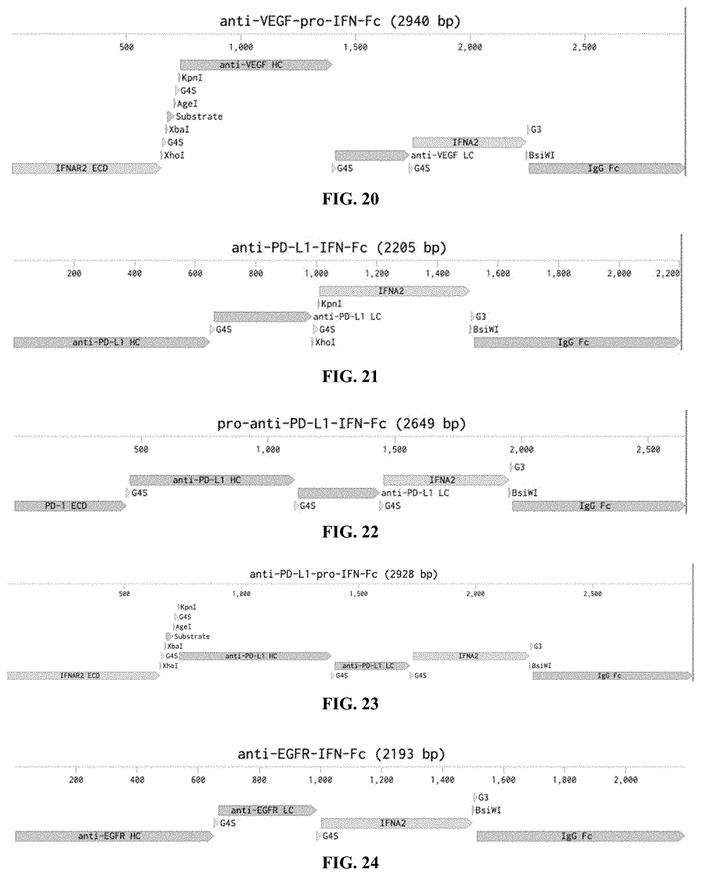

FIGS. 9A to 9E are graphs that show that Pro-IFN (SEQ ID NO:39) can suppress influenza-induced inflammation and infection in mice. FIG. 9A shows the expression of lung IL-6, FIG. 9B shows the expression of lung MCP-1, FIG. 9C shows the expression of H1N1 RNA normalized to GAPDH, FIG. 9D shows the expression of lung TNF, and FIG. 9E shows the expression of lung IFN-gamma.

For constructs involving anti-viral-associated or anti-epithelial-associated antibody fusion constructs, the following prophetic experiments are described:

During infection and inflammation, biomarkers such as PD-L1 will be upregulated, therefore anti-PD-L1-IFN will direct the IFN fusion protein into infectious sites. IFN further upregulates PD-L1 on all cells at the infectious site, allowing fusion proteins to retain in those tissues for greater efficacy and protective use.

In vitro Pseudotyped Viral Inhibition Assay. 2.5×10⁴ cells/well of 293-ACE2 or 1.0×10⁴ cells/well of VERO cells are seeded in 96-well plates. 1-day later, cells are incubated with IFN-Fc, anti-PD-L1-IFN-Fc, anti-VEGF-IFN-Fc, anti-EGFR-IFN-Fc, anti-PD-L1-IFN-Fc, PD-L1-pro-IFN-Fc, anti-VEGF-pro-IFN-Fc, and anti-EGFR-pro-IFN-Fc with and without MMP digestion.

SARS-CoV-2 or VSV pseudotyped lentiviral particles are inoculated on 293-ACE2 monolayers in the presence of 10 μg/ml of polybrene (Sigma-Aldrich) at the MOI of 0.01 or 0.5, respectively, and further incubated at 37° C. for 48-hours. VSV pseudotyped lentiviral particles are inoculated on VERO monolayers in the presence of 10 μg/ml of polybrene (Sigma-Aldrich) at a MOI of 0.75, and further incubated at 37° C. for 48-hours. IRFP reporter activity are measured on a CytoFLEX S Flow Cytometer (Beckman Coulter). The relative infection is calculated as the IRFP⁺ cell post-infection.

VERO and ACE2 293 cell lines are pre-treated with IFN-Fc, anti-PD-L1-IFN-Fc, anti-VEGF-IFN-Fc, anti-EGFR-IFN-Fc, anti-PD-L1-IFN-Fc, PD-L1-pro-IFN-Fc, anti-VEGF-pro-IFN-Fc, and anti-EGFR-pro-IFN-Fc with and without MMP digestion. 2 hours later, cells are infected with VSV-G. Viral load is measured 2 days later. Relative viral infection levels of VSV-G are reported by RFP or luciferase.

In vivo—Various doses of IFN-Fc, anti-PD-L1-IFN-Fc, anti-VEGF-IFN-Fc, anti-EGFR-IFN-Fc, anti-PD-L1-IFN-Fc, PD-L1-pro-IFN-Fc, anti-VEGF-pro-IFN-Fc, and anti-EGFR-pro-IFN-Fc are administered intranasally at 50 ul of 50, 150, 450 ug/ml on 4, 24, or 48 hours after viral infection. Lung tissues will be collected for determining viral titer and cytokine level as well as pathology. Cytokine levels are determined by cytokine bead array. Tissue RNA are collected and measured, and the expression of H1N1 RNA is normalized to GAPDH. 50 μl at different doses of the fusion protein are inhaled at 50 ug/ml, 150 ug/ml, and 450 ug/ml at 4, 24, and 48 hours after viral infection. Airway, nasal and lung tissues are collected on day 1, 3 and 7 post-infection. Tissues are digested for to ascertain viral titer and cytokine levels, as well as pathology. The cytokines will be determined by cytokine bead arrays. Tissues RNA will be collected and the expression of H1N1 RNA will be normalized to GAPDH.

Subject: human or a non-human mammal.

Preferred Method: inhalational use of nebulized or aerosolized drug into nasal and respiratory tract for controlling or preventing respiratory viral infection.

Secondary Method: intravenous

Exemplary Component Sequences of the mature protein.

An exemplary Signal Peptide is: LLSQNAFIFRSLNLVLMVYISLVFG—SEQ ID NO:17.

Pro-IFN composition is adapted from US 2020/0123227A1:

Components: The IFN prodrug contains: one or two copies of the type 1 IFN domain; or more than two copies of the type 1 IFN domain. The first linker is cleavable by at least one matrix metalloproteinases comprising matrix metalloproteinase MMP9 or MMP14. IgG1 Fc is fused to type 1 IFN, but other Fc domains may be used. The IFNAR is IFNAR1 or IFNAR2. The IFN can be any of: IFN-α, IFN-β, IFN-k, IFN-δ, IFN-ε, IFN-τ, IFN-ω or IFN-ξ. Other stabilizing proteins could also be used, including human serum albumin and transferrin. Heterodimeric constructs with different type 1 interferons fused to Fc are also possible.

| Mouse IFNAR1-ECD: | |
|---|---|
| SEQ ID NO: 1 | |
| 1 | MLAVVGAAAL VLVAGAPWVL PSAAGGENLK PPENIDVYII DDNYTLKWSS |
| | HGESMGSVTF |
| 61 | SAEYRTKDEA KWLKVPECQH TTTTKCEFSL LDTNVYIKTQ FRVRAEEGNS |
| | TSSWNEVDPF |
| 121 | IPFYTAHMSP PEVRLEAEDK AILVHISPPG QDGNMWALEK PSFSYTIRIW |
| | QKSSSDKKTI |
| 181 | NSTYYVEKIP ELLPETTYCL EVKAIHPSLK KHSNYSTVQC ISTTVANKMP |
| | VPGNLQVDAQ |
| 241 | GKSYVLKWDY IASADVLFRA QWLPGYSKSS SGSRSDKWKP IPTCANVQTT |
| | HCVFSQDTVY |
| 301 | TGTFFLHVQA SEGNHTSFWS EEKFIDSQKH ILPPPPVITV TAMSDTLLVY |
| | VNCQDSTCDG |
| 361 | LNYEIIFWEN TSNTKISMEK DGPEFTLKNL QPLTVYCVQA RVLFRALLNK |
| | TSNFSEKLCE |
| 421 | KTRPGSFST |
| Mouse IFNAR2-ECD: | |
| SEQ ID NO: 2 | |
| 1 | MRSRCTVSAV GLLSLCLVVS ASLETITPSA FDGYPDEPCT INITIRNSRL |
| | ILSWELENKS |
| 61 | GPPANYTLWY TVMSKDENLT KVKNCSDTTK SSCDVTDKWL EGMESYVVAI |
| | VIVHRGDLTV |
| 121 | CRCSDYIVPA NAPLEPPEFE IVGFTDHINV TMEFPPVTSK IIQEKMKTTP |
| | PVIKEQIGDS |
| 181 | VRKKHEPKVN NVTGNFTFVL RDLLPKTNYC VSLYFDDDPA IKSPLKCIVL |
| | QPGQESGLSE |
| 241 | SA |
| Human IFNAR1-ECD: | |
| SEQ ID NO: 3 | |
| 1 | MMVVLLGATT LVLVAVAPWV LSAAAGGKNL KSPQKVEVDI IDDNFILRWN |
| | RSDESVGNVT |
| 61 | FSFDYQKTGM DNWIKLSGCQ NITSTKCNFS SLKLNVYEEI KLRIRAEKEN |
| | TSSWYEVDSF |
| 121 | TPFRKAQIGP PEVHLEAEDK AIVIHISPGT KDSVMWALDG LSFTYSLVIW |
| | KNSSGVEERI |
| 181 | ENIYSRHKIY KLSPETTYCL KVKAALLTSW KIGVYSPVHC IKTTVENELP |
| | PPENIEVSVQ |
| 241 | NQNYVLKWDY TYANMTFQVQ WLHAFLKRNP GNHLYKWKQI PDCENVKTTQ |
| | CVFPQNVFQK |
| 301 | GIYLLRVQAS DGNNTSFWSE EIKFDTEIQA FLLPPVFNIR SLSDSFHIYI |
| | GAPKQSGNTP |
| 361 | VIQDYPLIYE IIFWENTSNA ERKIIEKKTD VTVPNLKPLT VYCVKARAHT |
| | MDEKLNKSSV |
| 421 | FSDAVCEKTK PGNTSK |
| Human IFNAR2-ECD: | |
| SEQ ID NO: 4 | |
| 1 | MLLSQNAFIF RSLNLVLMVY ISLVFGISYD SPDYTDESCT FKISLRNFRS |
| | ILSWELKNHS |
| 61 | IVPTHYTLLY TIMSKPEDLK VVKNCANTTR SFCDLTDEWR STHEAYVTVL |
| | EGFSGNTTLF |
| 121 | SCSHNFWLAI DMSFEPPEFE IVGFTNHINV MVKFPSIVEE ELQFDLSLVI |
| | EEQSEGIVKK |
| 181 | HKPEIKGNMS GNFTYIIDKL IPNTNYCVSV YLEHSDEQAV IKSPLKCTLL |
| | PPGQESESAE |
| 241 | SAK |

The domains above are linked together by short peptide linkers. The linker between the interferon molecule and IFNAR domain is subject to cleavage when the prodrug arrives at or near host cells infected by virus, thereby liberating the interferon molecule to act in the virally infected site to activate immune pathways and stimulate antigen presentation. Below are examples of enzymes overexpressed in viral infection, in particular respiratory infections such as SARS and influenza.

There are many MMP cleavable sequences in the public domain, some of which can be cleaved by multiple specificities. Exemplary substrate sequences include:

| Exemplary Protease | | |
|---|---|---|
| Substrates Enzyme | Substrate AA Sequence | |
| MMP2, MMP9 | PVGLIG | SEQ ID NO: 5 |
| MMP14 | SGRSENIRTA | SEQ ID NO: 6 |
| MMP14 | SGRSPAIFTA | SEQ ID NO: 18 |

Type 1 Interferon. IFN can be any of: IFN-α, IFN-β, IFN-k, IFN-δ, IFN-ε, IFN-τ, IFN-ω or IFN-ξ

Human IFN-α1, sp|P01562|24-189
SEQ ID NO: 7
CDLPETHSLDNRRTLMLLAQMSRISPSSCLMDRHDFGFPQEEFDGNQFQK
APAISVLHELIQQIFNLFTTKDSSAAWDEDLLDKFCTELYQQLNDLEACV
MQEERVGETPLMNADSILAVKKYFRRITLYLTEKKYSPCAWEVVRAEIMR
SLSLSTNLQERLRRKE
Human IFN-α2
SEQ ID NO: 8
CDLPQTHSLGSRRTLMLLAQMRRISLFSCLKDRHDFGFPQEEFGNQFQKA
ETIPVLHEMIQQIFNLFSTKDSSAAWDETLLDKFYTELYQQLNDLEACVI
QGVGVTETPLMKEDSILAVRKYFQRITLYLKEKKYSPCAWEVVRAEIMRS
FSLSTNLQESLRSKE

Also claimed are homodimer and heterodimer combinations with targeting antibody moieties such as anti-EGFR, anti-PD-L1 and anti-VEGF. The light chain and heavy chains are linked in scFv format, which can be connected by any type of non-cleavable linker, such as (GGGGS)ₙ, with sufficient length to permit scFv conformation.

Human Anti-PD-L1 scFv

Example: Atezolizumab

anti-PD-L1 heavy chain variable region
SEQ ID NO: 9
EVQLVESGGGLVQPGGSLRLSCAASGFTFSDSWIHWVRQAPGKGLEWVAW
ISPYGGSTYYADSVKGRFTISADTSKNTAYLQMNSLRAEDTAVYYCARRH
WPGGFDYWGQGTLVTVSSASTKGPSVFPLAPSSKSTSGGTAALGCLVKDY
FPEPVTVSWNSGALTSGVHTFPAVLQSSGLYSLSSVVTVPSSSLGTQTYI
CNVNHKPSNTKVDKKV
anti-PD-L1 light chain variable region
SEQ ID NO: 10
DIQMTQSPSSLSASVGDRVTITCRASQDVSTAVAWYQQKPGKAPKLLIYS
ASFLYSGVPSRFSGSGSGTDFTLTISSLQPEDFATYYCQQYLYHPATFGQ
GTKVEIK

Human Anti-EGFR scFv

Example: Cetuximab

anti-EGFR heavy chain variable region
SEQ ID NO: 11
QVQLKQSGPGLVQPSQSLSITCTVSGFSLTNYGVHWVRQSPGKGLEWLGV
IWSGGNTDYNTPFTSRLSINKDNSKSQVFFKMNSLQSNDTAIYYCARALT
YYDYEFAYWGQGTLVTVSAASTKGPSVFPLAPSSKSTSGGTAALGCLVKD
YFPEPVTVSWNSGALTSGVHTFPAVLQSSGLYSLSSVVTVPSSSLGTQTY
ICNVNHKPSNTKVDKKV
>anti-EGFR light chain variable region
SEQ ID NO: 12
DILLTQSPVILSVSPGERVSFSCRASQSIGTNIHWYQQRTNGSPRLLIKY
ASESISGIPSRFSGSGSGTDFTLSINSVESEDIADYYCQQNNNWPTTFGA
GTKLELK

Human Anti-VEGF scFv

Example: Bevacizumab

anti-VEGF heavy chain variable region
SEQ ID NO: 13
EVQLVESGGGLVQPGGSLRLSCAASGYTFTNYGMNWVRQAPGKGLEWVGW
INTYTGEPTYAADFKRRFTFSLDTSKSTAYLQMNSLRAEDTAVYYCAKYP
HYYGSSHWYFDVWGQGTLVTVSSASTKGPSVFPLAPSSKSTSGGTAALGC
LVKDYFPEPVTVSWNSGALTSGVHTFPAVLQSSGLYSLSSVVTVPSSSLG
TQTYICNVNHKPSNTKVDKKV
anti-VEGF light chain variable region
SEQ ID NO: 14
DIQMTQSPSSLSASVGDRVTITCSASQDISNYLNWYQQKPGKAPKVLIYF
TSSLHSGVPSRFSGSGSGTDFTLTISSLQPEDFATYYCQQYSTVPWTFGQ
GTKVEIK

The scFv antibodies are linked to pro-IFN via cleavable or non-cleavable linkers. The targeting antibodies may be linked to blocking moieties as well via the same cleavable linkers as specified above.

For anti-PD-L1, the blocking moiety is the extracellular domain of PD-1.

sp|Q15116|24-170
SEQ ID NO: 15
FLDSPDRPWNPPTFSPALLVVTEGDNATFTCSFSNTSESFVLNWYRMSPS
NQTDKLAAFPEDRSQPGQDCRFRVTQLPNGRDFHMSVVRARRNDSGTYLC
GAISLAPKAQIKESLRAELRVTERRAEVPTAHPSPSPRPAGQFQTLV

For anti-VEGF, the blocking moiety is isoform VEGF165B or a subsequence thereof that is capable of binding to the scFv. Isoform VEGF165B does not activate downstream signaling pathways and does not activate angiogenesis.

sp|P15692-8|VEGFA_HUMAN Isoform VEGF165B of
Vascular endothelial growth factor A OS = Homo
sapiens OX = 9606 GN = VEGFA
SEQ ID NO: 16
MNFLLSWVHWSLALLLYLHHAKWSQAAPMAEGGGQNHHEVVKFMDVYQRS
YCHPIETLVDIFQEYPDEIEYIFKPSCVPLMRCGGCCNDEGLECVPTEES
NITMQIMRIKPHQGQHIGEMSFLQHNKCECRPKKDRARQENPCGPCSERR
KHLFVQDPQTCKCSCKNTDSRCKARQLELNERTCRSLTRKD

Pro-IFN and IFN Fusion Construct Sequences and Maps.

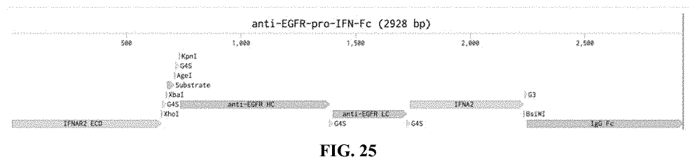

FIG. 10 is a map of a Pro-IFN-Fc fusion protein of SEQ ID NO:19.

ISYDSPDYTDESCTFKISLRNFRSILSWELKNHSIVPTHYTLLYTIMSKP
EDLKVVKNCANTTRSFCDLTDEWRSTHEAYVTVLEGFSGNTTLFSCSHNF
WLAIDMSFEPPEFEIVGFTNHINVMVKFPSIVEEELQFDLSLVIEEQSEG
IVKKHKPEIKGNMSGNFTYIIDKLIPNTNYCVSVYLEHSDEQAVIKSPLK
CTLLPPGQESESAESAKLEGGGGSSRSGRSPAIFTATGGGGGSGTCDLPQ
THSLGSRRTLMLLAQMRRISLFSCLKDRHDFGFPQEEFGNQFQKAETIPV
LHEMIQQIFNLFSTKDSSAAWDETLLDKFYTELYQQLNDLEACVIQGVGV
TETPLMKEDSILAVRKYFQRITLYLKEKKYSPCAWEVVRAEIMRSFSLST
NLQESLRSKERTGGGDKTHTCPPCPAPELLGGPSVFLFPPKPKDTLMISR
TPEVTCVVVDVSHEDPEVKFNWYVDGVEVHNAKTKPREEQYNSTYRVVSV
LTVLHQDWLNGKEYKCKVSNKALPAPIEKTISKAKGQPREPQVYTLPPSR
DELTKNQVSLTCLVKGFYPSDIAVEWESNGQPENNYKTTPPVLDSDGSFF
LYSKLTVDKSRWQQGNVFSCSVMHEALHNHYTQKSLSLSPGK

Exemplary Heterodimer Constructs—all Fc6 and Fc9 Combinations

FIG. 11 is a map of an Anti-VEGF-Fc6 fusion protein of SEQ ID NO: 20.

EVQLVESGGGLVQPGGSLRLSCAASGYTFTNYGMNWVRQAPGKGLEWVGW
INTYTGEPTYAADFKRRFTFSLDTSKSTAYLQMNSLRAEDTAVYYCAKYP
HYYGSSHWYFDVWGQGTLVTVSSASTKGPSVFPLAPSSKSTSGGTAALGC
LVKDYFPEPVTVSWNSGALTSGVHTFPAVLQSSGLYSLSSVVTVPSSSLG
TQTYICNVNHKPSNTKVDKKVGGGGSDIQMTQSPSSLSASVGDRVTITCS
ASQDISNYLNWYQQKPGKAPKVLIYFTSSLHSGVPSRFSGSGSGTDFTLT
ISSLQPEDFATYYCQQYSTVPWTFGQGTKVEIKRTGGGDKTHTCPPCPAP
ELLGGPSVFLFPPKPKDTLMISRTPEVTCVVVDVSHEDPEVKFNWYVDGV
EVHNAKTKPREEQYNSTYRVVSVLTVLHQDWLNGKEYKCKVSNKALPAPI
EKTISKAKGQPREPQVCTLPPSRDELTKNQVSLSCAVKGFYPSDIAVEWE
SNGQPENNYKTTPPVLDSDGSFFLVSKLTVDKSRWQQGNVFSCSVMHEAL
HNHYTQKSLSLSPGK

FIG. 12 is a map of a Pro-anti-VEGF-Fc6 fusion protein of SEQ ID NO: 21.

MNFLLSWVHWSLALLLYLHHAKWSQAAPMAEGGGQNHHEVVKFMDVYQRS
YCHPIETLVDIFQEYPDEIEYIFKPSCVPLMRCGGCCNDEGLECVPTEES
NITMQIMRIKPHQGQHIGEMSFLQHNKCECRPKKDRARQENPCGPCSERR
KHLFVQDPQTCKCSCKNTDSRCKARQLELNERTCRSLTRKDLEGGGGSSR
SGRSPAIFTATGGGGGSGTEVQLVESGGGLVQPGGSLRLSCAASGYTFTN
YGMNWVRQAPGKGLEWVGWINTYTGEPTYAADFKRRFTFSLDTSKSTAYL
QMNSLRAEDTAVYYCAKYPHYYGSSHWYFDVWGQGTLVTVSSASTKGPSV
FPLAPSSKSTSGGTAALGCLVKDYFPEPVTVSWNSGALTSGVHTFPAVLQ
SSGLYSLSSVVTVPSSSLGTQTYICNVNHKPSNTKVDKKVGGGGSDIQMT
QSPSSLSASVGDRVTITCSASQDISNYLNWYQQKPGKAPKVLIYFTSSLH
SGVPSRFSGSGSGTDFTLTISSLQPEDFATYYCQQYSTVPWTFGQGTKVE
IKRTGGGDKTHTCPPCPAPELLGGPSVFLFPPKPKDTLMISRTPEVTCVV
VDVSHEDPEVKFNWYVDGVEVHNAKTKPREEQYNSTYRVVSVLTVLHQDW
LNGKEYKCKVSNKALPAPIEKTISKAKGQPREPQVCTLPPSRDELTKNQV
SLSCAVKGFYPSDIAVEWESNGQPENNYKTTPPVLDSDGSFFLVSKLTVD
KSRWQQGNVFSCSVMHEALHNHYTQKSLSLSPGK

FIG. 13 is a map of an Anti-PD-L1-Fc6 fusion protein of SEQ ID NO:22.

EVQLVESGGGLVQPGGSLRLSCAASGFTFSDSWIHWVRQAPGKGLEWVAW
ISPYGGSTYYADSVKGRFTISADTSKNTAYLQMNSLRAEDTAVYYCARRH
WPGGFDYWGQGTLVTVSSASTKGPSVFPLAPSSKSTSGGTAALGCLVKDY
FPEPVTVSWNSGALTSGVHTFPAVLQSSGLYSLSSVVTVPSSSLGTQTYI
CNVNHKPSNTKVDKKVGGGGSDIQMTQSPSSLSASVGDRVTITCRASQDV
STAVAWYQQKPGKAPKLLIYSASFLYSGVPSRFSGSGSGTDFTLTISSLQ
PEDFATYYCQQYLYHPATFGQGTKVEIKRTGGGDKTHTCPPCPAPELLGG
PSVFLFPPKPKDTLMISRTPEVTCVVVDVSHEDPEVKFNWYVDGVEVHNA
KTKPREEQYNSTYRVVSVLTVLHQDWLNGKEYKCKVSNKALPAPIEKTIS
KAKGQPREPQVCTLPPSRDELTKNQVSLSCAVKGFYPSDIAVEWESNGQP
ENNYKTTPPVLDSDGSFFLVSKLTVDKSRWQQGNVFSCSVMHEALHNHYT
QKSLSLSPGK

FIG. 14 is a map of a Pro-anti-PD-L1-Fc6 fusion protein of SEQ ID NO:23.

FLDSPDRPWNPPTFSPALLVVTEGDNATFTCSFSNTSESFVLNWYRMSPS
NQTDKLAAFPEDRSQPGQDCRFRVTQLPNGRDFHMSVVRARRNDSGTYLC
GAISLAPKAQIKESLRAELRVTERRAEVPTAHPSPSPRPAGQFQTLVGGG
GSEVQLVESGGGLVQPGGSLRLSCAASGFTFSDSWIHWVRQAPGKGLEWV
AWISPYGGSTYYADSVKGRFTISADTSKNTAYLQMNSLRAEDTAVYYCAR
RHWPGGFDYWGQGTLVTVSSASTKGPSVFPLAPSSKSTSGGTAALGCLVK
DYFPEPVTVSWNSGALTSGVHTFPAVLQSSGLYSLSSVVTVPSSSLGTQT
YICNVNHKPSNTKVDKKVGGGGSDIQMTQSPSSLSASVGDRVTITCRASQ
DVSTAVAWYQQKPGKAPKLLIYSASFLYSGVPSRFSGSGSGTDFTLTISS
LQPEDFATYYCQQYLYHPATFGQGTKVEIKGGGGSDKTHTCPPCPAPELL
GGPSVFLFPPKPKDTLMISRTPEVTCVVVDVSHEDPEVKFNWYVDGVEVH
NAKTKPREEQYNSTYRVVSVLTVLHQDWLNGKEYKCKVSNKALPAPIEKT
ISKAKGQPREPQVCTLPPSRDELTKNQVSLSCAVKGFYPSDIAVEWESNG
QPENNYKTTPPVLDSDGSFFLVSKLTVDKSRWQQGNVFSCSVMHEALHNH
YTQKSLSLSPGK

FIG. 15 is a map of an Anti-EGFR-Fc6 fusion protein of SEQ ID NO:24.

QVQLKQSGPGLVQPSQSLSITCTVSGFSLTNYGVHWVRQSPGKGLEWLGV
IWSGGNTDYNTPFTSRLSINKDNSKSQVFFKMNSLQSNDTAIYYCARALT
YYDYEFAYWGQGTLVTVSAASTKGPSVFPLAPSSKSTSGGTAALGCLVKD
YFPEPVTVSWNSGALTSGVHTFPAVLQSSGLYSLSSVVTVPSSSLGTQTY
ICNVNHKPSNTKVDKKVGGGGSDILLTQSPVILSVSPGERVSFSCRASQS
IGTNIHWYQQRTNGSPRLLIKYASESISGIPSRFSGSGSGTDFTLSINSV
ESEDIADYYCQQNNNWPTTFGAGTKLELKRTGGGDKTHTCPPCPAPELLG
GPSVFLFPPKPKDTLMISRTPEVTCVVVDVSHEDPEVKFNWYVDGVEVHN
AKTKPREEQYNSTYRVVSVLTVLHQDWLNGKEYKCKVSNKALPAPIEKTI
SKAKGQPREPQVCTLPPSRDELTKNQVSLSCAVKGFYPSDIAVEWESNGQ
PENNYKTTPPVLDSDGSFFLVSKLTVDKSRWQQGNVFSCSVMHEALHNHY
TQKSLSLSPGK

FIG. 16 is a map of an IFN-Fc9 fusion protein of SEQ ID NO:25.

CDLPQTHSLGSRRTLMLLAQMRRISLFSCLKDRHDFGFPQEEFGNQFQKA
ETIPVLHEMIQQIFNLFSTKDSSAAWDETLLDKFYTELYQQLNDLEACVI
QGVGVTETPLMKEDSILAVRKYFQRITLYLKEKKYSPCAWEVVRAEIMRS
FSLSTNLQESLRSKERTGGGDKTHTCPPCPAPELLGGPSVFLFPPKPKDT
LMISRTPEVTCVVVDVSHEDPEVKFNWYVDGVEVHNAKTKPREEQYNSTY
RVVSVLTVLHQDWLNGKEYKCKVSNKALPAPIEKTISKAKGQPREPQVYT
LPPCRDELTKNQVSLWCLVKGFYPSDIAVEWESNGQPENNYKTTPPVLDS
DGSFFLYSKLTVDKSRWQQGNVFSCSVMHEALHNHYTQKSLSLSPGK

FIG. 17 is a ma of a Pro-IFN-Fc9 fusion protein of SE ID NO:26.

ISYDSPDYTDESCTFKISLRNFRSILSWELKNHSIVPTHYTLLYTIMSKP
EDLKVVKNCANTTRSFCDLTDEWRSTHEAYVTVLEGFSGNTTLFSCSHNF
WLAIDMSFEPPEFEIVGFTNHINVMVKFPSIVEEELQFDLSLVIEEQSEG
IVKKHKPEIKGNMSGNFTYIIDKLIPNTNYCVSVYLEHSDEQAVIKSPLK
CTLLPPGQESESAESAKLEGGGGSSRSGRSPAIFTATGGGGGSGTCDLPQ
THSLGSRRTLMLLAQMRRISLFSCLKDRHDFGFPQEEFGNQFQKAETIPV
LHEMIQQIFNLFSTKDSSAAWDETLLDKFYTELYQQLNDLEACVIQGVGV
TETPLMKEDSILAVRKYFQRITLYLKEKKYSPCAWEVVRAEIMRSFSLST
NLQESLRSKERTGGGDKTHTCPPCPAPELLGGPSVFLFPPKPKDTLMISR
TPEVTCVVVDVSHEDPEVKFNWYVDGVEVHNAKTKPREEQYNSTYRVVSV
LTVLHQDWLNGKEYKCKVSNKALPAPIEKTISKAKGQPREPQVYTLPPCR
DELTKNQVSLWCLVKGFYPSDIAVEWESNGQPENNYKTTPPVLDSDGSFF
LYSKLTVDKSRWQQGNVFSCSVMHEALHNHYTQKSLSLSPGK

Exemplary Homodimer Constructs.

FIG. 18 is a ma of an Anti-VEGF-IFN-Fc fusion protein of SE ID NO:27.

EVQLVESGGGLVQPGGSLRLSCAASGYTFTNYGMNWVRQAPGKGLEWVGW
INTYTGEPTYAADFKRRFTFSLDTSKSTAYLQMNSLRAEDTAVYYCAKYP
HYYGSSHWYFDVWGQGTLVTVSSASTKGPSVFPLAPSSKSTSGGTAALGC
LVKDYFPEPVTVSWNSGALTSGVHTFPAVLQSSGLYSLSSVVTVPSSSLG
TQTYICNVNHKPSNTKVDKKVGGGGSDIQMTQSPSSLSASVGDRVTITCS
ASQDISNYLNWYQQKPGKAPKVLIYFTSSLHSGVPSRFSGSGSGTDFTLT
ISSLQPEDFATYYCQQYSTVPWTFGQGTKVEIKGGGGSCDLPQTHSLGSR
RTLMLLAQMRRISLFSCLKDRHDFGFPQEEFGNQFQKAETIPVLHEMIQQ
IFNLFSTKDSSAAWDETLLDKFYTELYQQLNDLEACVIQGVGVTETPLMK
EDSILAVRKYFQRITLYLKEKKYSPCAWEVVRAEIMRSFSLSTNLQESLR
SKERTGGGDKTHTCPPCPAPELLGGPSVFLFPPKPKDTLMISRTPEVTCV
VVDVSHEDPEVKFNWYVDGVEVHNAKTKPREEQYNSTYRVVSVLTVLHQD
WLNGKEYKCKVSNKALPAPIEKTISKAKGQPREPQVYTLPPSRDELTKNQ
VSLTCLVKGFYPSDIAVEWESNGQPENNYKTTPPVLDSDGSFFLYSKLTV
DKSRWQQGNVFSCSVMHEALHNHYTQKSLSLSPGK

FIG. 19 is a map of a Pro-anti-VEGF-IFN-Fc fusion protein of SEQ ID NO:28.

MNFLLSWVHWSLALLLYLHHAKWSQAAPMAEGGGQNHHEVVKFMDVYQRS
YCHPIETLVDIFQEYPDEIEYIFKPSCVPLMRCGGCCNDEGLECVPTEES
NITMQIMRIKPHQGQHIGEMSFLQHNKCECRPKKDRARQENPCGPCSERR
KHLFVQDPQTCKCSCKNTDSRCKARQLELNERTCRSLTRKDLEGGGGSSR
SGRSPAIFTATGGGGGSGTEVQLVESGGGLVQPGGSLRLSCAASGYTFTN
YGMNWVRQAPGKGLEWVGWINTYTGEPTYAADFKRRFTFSLDTSKSTAYL
QMNSLRAEDTAVYYCAKYPHYYGSSHWYFDVWGQGTLVTVSSASTKGPSV
FPLAPSSKSTSGGTAALGCLVKDYFPEPVTVSWNSGALTSGVHTFPAVLQ
SSGLYSLSSVVTVPSSSLGTQTYICNVNHKPSNTKVDKKVGGGGSDIQMT
QSPSSLSASVGDRVTITCSASQDISNYLNWYQQKPGKAPKVLIYFTSSLH
SGVPSRFSGSGSGTDFTLTISSLQPEDFATYYCQQYSTVPWTFGQGTKVE
IKCDLPQTHSLGSRRTLMLLAQMRRISLFSCLKDRHDFGFPQEEFGNQFQ
KAETIPVLHEMIQQIFNLFSTKDSSAAWDETLLDKFYTELYQQLNDLEAC
VIQGVGVTETPLMKEDSILAVRKYFQRITLYLKEKKYSPCAWEVVRAEIM
RSFSLSTNLQESLRSKERTGGGDKTHTCPPCPAPELLGGPSVFLFPPKPK
DTLMISRTPEVTCVVVDVSHEDPEVKFNWYVDGVEVHNAKTKPREEQYNS
TYRVVSVLTVLHQDWLNGKEYKCKVSNKALPAPIEKTISKAKGQPREPQV
YTLPPSRDELTKNQVSLT

FIG. 20 is a map of an Anti-VEGF-pro-IFN-Fc fusion protein of SEQ ID NO:29.

ISYDSPDYTDESCTFKISLRNFRSILSWELKNHSIVPTHYTLLYTIMSKP
EDLKVVKNCANTTRSFCDLTDEWRSTHEAYVTVLEGFSGNTTLFSCSHNF
WLAIDMSFEPPEFEIVGFTNHINVMVKFPSIVEEELQFDLSLVIEEQSEG
IVKKHKPEIKGNMSGNFTYIIDKLIPNTNYCVSVYLEHSDEQAVIKSPLK
CTLLPPGQESESAESAKLEGGGGSSRSGRSPAIFTATGGGGGSGTEVQLV
ESGGGLVQPGGSLRLSCAASGYTFTNYGMNWVRQAPGKGLEWVGWINTYT
GEPTYAADFKRRFTFSLDTSKSTAYLQMNSLRAEDTAVYYCAKYPHYYGS
SHWYFDVWGQGTLVTVSSASTKGPSVFPLAPSSKSTSGGTAALGCLVKDY
FPEPVTVSWNSGALTSGVHTFPAVLQSSGLYSLSSVVTVPSSSLGTQTYI
CNVNHKPSNTKVDKKVGGGGSDIQMTQSPSSLSASVGDRVTITCSASQDI
SNYLNWYQQKPGKAPKVLIYFTSSLHSGVPSRFSGSGSGTDFTLTISSLQ
PEDFATYYCQQYSTVPWTFGQGTKVEIKGGGGSCDLPQTHSLGSRRTLML
LAQMRRISLFSCLKDRHDFGFPQEEFGNQFQKAETIPVLHEMIQQIFNLF
STKDSSAAWDETLLDKFYTELYQQLNDLEACVIQGVGVTETPLMKEDSIL
AVRKYFQRITLYLKEKKYSPCAWEVVRAEIMRSFSLSTNLQESLRSKERT
GGGDKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPQVYTLPPSRDELTKNQVSLTC
LVKGFYPSDIAVEWESNGQPENNYKTTPPVLDSDGSFFLYSKLTVDKSRW
QQGNVFSCSVMHEALHNHYTQKSLSLSPGK

FIG. 21 is a map of an Anti-PD-L1-IFN-Fc fusion protein of SEQ ID NO:30.

EVQLVESGGGLVQPGGSLRLSCAASGFTFSDSWIHWVRQAPGKGLEWVAW
ISPYGGSTYYADSVKGRFTISADTSKNTAYLQMNSLRAEDTAVYYCARRH
WPGGFDYWGQGTLVTVSSASTKGPSVFPLAPSSKSTSGGTAALGCLVKDY
FPEPVTVSWNSGALTSGVHTFPAVLQSSGLYSLSSVVTVPSSSLGTQTYI
CNVNHKPSNTKVDKKVGGGGSDIQMTQSPSSLSASVGDRVTITCRASQDV
STAVAWYQQKPGKAPKLLIYSASFLYSGVPSRFSGSGSGTDFTLTISSLQ
PEDFATYYCQQYLYHPATFGQGTKVEIKLEGGGGSGTCDLPQTHSLGSRR
TLMLLAQMRRISLFSCLKDRHDFGFPQEEFGNQFQKAETIPVLHEMIQQI
FNLFSTKDSSAAWDETLLDKFYTELYQQLNDLEACVIQGVGVTETPLMKE
DSILAVRKYFQRITLYLKEKKYSPCAWEVVRAEIMRSFSLSTNLQESLRS
KERTGGGDKTHTCPPCPAPELLGGPSVFLFPPKPKDTLMISRTPEVTCVV
VDVSHEDPEVKFNWYVDGVEVHNAKTKPREEQYNSTYRVVSVLTVLHQDW
LNGKEYKCKVSNKALPAPIEKTISKAKGQPREPQVYTLPPSRDELTKNQV
SLTCLVKGFYPSDIAVEWESNGQPENNYKTTPPVLDSDGSFFLYSKLTVD
KSRWQQGNVFSCSVMHEALHNHYTQKSLSLSPGK

FIG. 22 is a map of a Pro-anti-PD-L1-IFN-Fc fusion protein of SEQ ID NO:31.

FLDSPDRPWNPPTFSPALLVVTEGDNATFTCSFSNTSESFVLNWYRMSPS
NQTDKLAAFPEDRSQPGQDCRFRVTQLPNGRDFHMSVVRARRNDSGTYLC
GAISLAPKAQIKESLRAELRVTERRAEVPTAHPSPSPRPAGQFQTLVGGG
GSEVQLVESGGGLVQPGGSLRLSCAASGFTFSDSWIHWVRQAPGKGLEWV
AWISPYGGSTYYADSVKGRFTISADTSKNTAYLQMNSLRAEDTAVYYCAR
RHWPGGFDYWGQGTLVTVSSASTKGPSVFPLAPSSKSTSGGTAALGCLVK
DYFPEPVTVSWNSGALTSGVHTFPAVLQSSGLYSLSSVVTVPSSSLGTQT
YICNVNHKPSNTKVDKKVGGGGSDIQMTQSPSSLSASVGDRVTITCRASQ
DVSTAVAWYQQKPGKAPKLLIYSASFLYSGVPSRFSGSGSGTDFTLTISS
LQPEDFATYYCQQYLYHPATFGQGTKVEIKGGGGSCDLPQTHSLGSRRTL
MLLAQMRRISLFSCLKDRHDFGFPQEEFGNQFQKAETIPVLHEMIQQIFN
LFSTKDSSAAWDETLLDKFYTELYQQLNDLEACVIQGVGVTETPLMKEDS
ILAVRKYFQRITLYLKEKKYSPCAWEVVRAEIMRSFSLSTNLQESLRSKE
RTGGGDKTHTCPPCPAPELLGGPSVFLFPPKPKDTLMISRTPEVTCVVVD
VSHEDPEVKFNWYVDGVEVHNAKTKPREEQYNSTYRVVSVLTVLHQDWLN
GKEYKCKVSNKALPAPIEKTISKAKGQPREPQVYTLPPSRDELTKNQVSL
TCLVKGFYPSDIAVEWESNGQPENNYKTTPPVLDSDGSFFLYSKLTVDKS
RWQQGNVFSCSVMHEALHNHYTQKSLSLSPGK

FIG. 23 is a map of an Anti-PD-L1-pro-IFN-Fc fusion protein of SEQ ID NO:32.

ISYDSPDYTDESCTFKISLRNFRSILSWELKNHSIVPTHYTLLYTIMSKP
EDLKVVKNCANTTRSFCDLTDEWRSTHEAYVTVLEGFSGNTTLFSCSHNF
WLAIDMSFEPPEFEIVGFTNHINVMVKFPSIVEEELQFDLSLVIEEQSEG
IVKKHKPEIKGNMSGNFTYIIDKLIPNTNYCVSVYLEHSDEQAVIKSPLK
CTLLPPGQESESAESAKLEGGGGSSRSGRSPAIFTATGGGGGSGTEVQLV
ESGGGLVQPGGSLRLSCAASGFTFSDSWIHWVRQAPGKGLEWVAWISPYG
GSTYYADSVKGRFTISADTSKNTAYLQMNSLRAEDTAVYYCARRHWPGGF
DYWGQGTLVTVSSASTKGPSVFPLAPSSKSTSGGTAALGCLVKDYFPEPV
TVSWNSGALTSGVHTFPAVLQSSGLYSLSSVVTVPSSSLGTQTYICNVNH
KPSNTKVDKKVGGGGSDIQMTQSPSSLSASVGDRVTITCRASQDVSTAVA
WYQQKPGKAPKLLIYSASFLYSGVPSRFSGSGSGTDFTLTISSLQPEDFA
TYYCQQYLYHPATFGQGTKVEIKGGGGSCDLPQTHSLGSRRTLMLLAQMR
RISLFSCLKDRHDFGFPQEEFGNQFQKAETIPVLHEMIQQIFNLFSTKDS
SAAWDETLLDKFYTELYQQLNDLEACVIQGVGVTETPLMKEDSILAVRKY
FQRITLYLKEKKYSPCAWEVVRAEIMRSFSLSTNLQESLRSKERTGGGDK
THTCPPCPAPELLGGPSVFLFPPKPKDTLMISRTPEVTCVVVDVSHEDPE
VKFNWYVDGVEVHNAKTKPREEQYNSTYRVVSVLTVLHQDWLNGKEYKCK
VSNKALPAPIEKTISKAKGQPREPQVYTLPPSRDELTKNQVSLTCLVKGF
YPSDIAVEWESNGQPENNYKTTPPVLDSDGSFFLYSKLTVDKSRWQQGNV
FSCSVMHEALHNHYTQKSLSLSPGK

FIG. 24 is a map of an Anti-EGFR-IFN-Fc fusion protein of SEQ ID NO:33.

QVQLKQSGPGLVQPSQSLSITCTVSGFSLTNYGVHWVRQSPGKGLEWLGV
IWSGGNTDYNTPFTSRLSINKDNSKSQVFFKMNSLQSNDTAIYYCARALT
YYDYEFAYWGQGTLVTVSAASTKGPSVFPLAPSSKSTSGGTAALGCLVKD
YFPEPVTVSWNSGALTSGVHTFPAVLQSSGLYSLSSVVTVPSSSLGTQTY
ICNVNHKPSNTKVDKKVGGGGSDILLTQSPVILSVSPGERVSFSCRASQS
IGTNIHWYQQRTNGSPRLLIKYASESISGIPSRFSGSGSGTDFTLSINSV
ESEDIADYYCQQNNNWPTTFGAGTKLELKGGGGSCDLPQTHSLGSRRTLM
LLAQMRRISLFSCLKDRHDFGFPQEEFGNQFQKAETIPVLHEMIQQIFNL
FSTKDSSAAWDETLLDKFYTELYQQLNDLEACVIQGVGVTETPLMKEDSI
LAVRKYFQRITLYLKEKKYSPCAWEVVRAEIMRSFSLSTNLQESLRSKER
TGGGDKTHTCPPCPAPELLGGPSVFLFPPKPKDTLMISRTPEVTCVVVDV
SHEDPEVKFNWYVDGVEVHNAKTKPREEQYNSTYRVVSVLTVLHQDWLNG
KEYKCKVSNKALPAPIEKTISKAKGQPREPQVYTLPPSRDELTKNQVSLT
CLVKGFYPSDIAVEWESNGQPENNYKTTPPVLDSDGSFFLYSKLTVDKSR
WQQGNVFSCSVMHEALHNHYTQKSLSLSPGK

FIG. 25 is a map of an Anti-EGFR-pro-IFN-Fc fusion protein of SEQ ID NO:34.

ISYDSPDYTDESCTFKISLRNFRSILSWELKNHSIVPTHYTLLYTIMSKP
EDLKVVKNCANTTRSFCDLTDEWRSTHEAYVTVLEGFSGNTTLFSCSHNF
WLAIDMSFEPPEFEIVGFTNHINVMVKFPSIVEEELQFDLSLVIEEQSEG
IVKKHKPEIKGNMSGNFTYIIDKLIPNTNYCVSVYLEHSDEQAVIKSPLK
CTLLPPGQESESAESAKLEGGGGSSRSGRSPAIFTATGGGGGSGTQVQLK
QSGPGLVQPSQSLSITCTVSGFSLTNYGVHWVRQSPGKGLEWLGVIWSGG
NTDYNTPFTSRLSINKDNSKSQVFFKMNSLQSNDTAIYYCARALTYYDYE
FAYWGQGTLVTVSAASTKGPSVFPLAPSSKSTSGGTAALGCLVKDYFPEP
VTVSWNSGALTSGVHTFPAVLQSSGLYSLSSVVTVPSSSLGTQTYICNVN
HKPSNTKVDKKVGGGGSDILLTQSPVILSVSPGERVSFSCRASQSIGTNI
HWYQQRTNGSPRLLIKYASESISGIPSRFSGSGSGTDFTLSINSVESEDI
ADYYCQQNNNWPTTFGAGTKLELKGGGGSCDLPQTHSLGSRRTLMLLAQM
RRISLFSCLKDRHDFGFPQEEFGNQFQKAETIPVLHEMIQQIFNLFSTKD
SSAAWDETLLDKFYTELYQQLNDLEACVIQGVGVTETPLMKEDSILAVRK
YFQRITLYLKEKKYSPCAWEVVRAEIMRSFSLSTNLQESLRSKERTGGGD
KTHTCPPCPAPELLGGPSVFLFPPKPKDTLMISRTPEVTCVVVDVSHEDP
EVKFNWYVDGVEVHNAKTKPREEQYNSTYRVVSVLTVLHQDWLNGKEYKC
KVSNKALPAPIEKTISKAKGQPREPQVYTLPPSRDELTKNQVSLTCLVKG
FYPSDIAVEWESNGQPENNYKTTPPVLDSDGSFFLYSKLTVDKSRWQQGN
VFSCSVMHEALHNHYTQKSLSLSPGK

As described in US20200123227A1, an exemplary form of the cleavable linker is GGGGS-substrate-GGGGS SEQ ID NO: 35 or (GGGGS)ₙ-substrate-(GGGGS)ₙ SEQ ID NO: 36, where n could be any number. Non-cleavable linkers are (GGGGS) SEQ ID NO: 37.

IFN-Fc Sequence of SEQ ID NO:38.

CDLPQTHSLGSRRTLMLLAQMRRISLFSCLKDRHDFGFPQEEFGNQFQKA
ETIPVLHEMIQQIFNLFSTKDSSAAWDETLLDKFYTELYQQLNDLEACVI
QGVGVTETPLMKEDSILAVRKYFQRITLYLKEKKYSPCAWEVVRAEIMRS
FSLSTNLQESLRSKERTGGGDKTHTCPPCPAPELLGGPSVFLFPPKPKDT
LMISRTPEVTCVVVDVSHEDPEVKFNWYVDGVEVHNAKTKPREEQYNSTY
RVVSVLTVLHQDWLNGKEYKCKVSNKALPAPIEKTISKAKGQPREPQVYT
LPPSRDELTKNQVSLTCLVKGFYPSDIAVEWESNGQPENNYKTTPPVLDS
DGSFFLYSKLTVDKSRWQQGNVFSCSVMHEALHNHYTQKSLSLSPGK

Mouse Pro-IFN Sequence of SEQ ID NO:39

MRSRCTVSAVGLLSLCLVVSASLETITPSAFDGYPDEPCTINITIRNSRL
ILSWELENKSGPPANYTLWYTVMSKDENLTKVKNCSDTTKSSCDVTDKWL
EGMESYVVAIVIVHRGDLTVCRCSDYIVPANAPLEPPEFEIVGFTDHINV
TMEFPPVTSKIIQEKMKTTPFVIKEQIGDSVRKKHEPKVNNVTGNFTFVL
RDLLPKTNYCVSLYFDDDPAIKSPLKCIVLQPGQESGLSESAGGGGSSRS
GRSPAIFTATGGGGGSGTCDLPHTYNLGNKRALTVLEEMRRLPPLSCLKD
RKDFGFPLEKVDNQQIQKAQAILVLRDLTQQILNLFTSKDLSATWNATLL
DSFCNDLHQQLNDLKACVMQEPPLTQEDSLLAVRTYFHRITVYLRKKKHS
LCAWEVIRAEVWRALSSSTNLLARLSEEKEGGGDKTHTCPPCPAPELLGG
PSVFLFPPKPKDTLMISRTPEVTCVVVDVSHEDPEVKFNWYVDGVEVHNA
KTKPREEQYNSTYRVVSVLTVLHQDWLNGKEYKCKVSNKALPAPIEKTIS
KAKGQPREPQVYTLPPSRDELTKNQVSLTCLVKGFYPSDIAVEWESNGQP
ENNYKTTPPVLDSDGSFFLYSKLTVDKSRWQQGNVFSCSVMHEALHNHYT
QKSLSLSPGK

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited features, elements, components, groups, integers, and/or steps, but do not exclude the presence of other unstated features, elements, components, groups, integers and/or steps. In embodiments of any of the compositions and methods provided herein, “comprising” may be replaced with “consisting essentially of” or “consisting of”. As used herein, the term “consisting” is used to indicate the presence of the recited integer (e.g., a feature, an element, a characteristic, a property, a method/process step or a limitation) or group of integers (e.g., feature(s), element(s), characteristic(s), property(ies), method/process steps or limitation(s)) only. As used herein, the phrase “consisting essentially of” requires the specified features, elements, components, groups, integers, and/or steps, but do not exclude the presence of other unstated features, elements, components, groups, integers and/or steps as well as those that do not materially affect the basic and novel characteristic(s) and/or function of the claimed invention.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skill in the art recognize the modified feature as still having the required characteristics and capabilities of the unmodified feature. In general, but subject to the preceding discussion, a numerical value herein that is modified by a word of approximation such as “about” may vary from the stated value by at least 1, 2, 3, 4, 5, 6, 7, 10, 12 or 15%.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

To aid the Patent Office, and any readers of any patent issued on this application in interpreting the claims appended hereto, applicants wish to note that they do not intend any of the appended claims to invoke paragraph 6 of 35 U.S.C. § 112, U.S.C. § 112 paragraph (f), or equivalent, as it exists on the date of filing hereof unless the words “means for” or “step for” are explicitly used in the particular claim.

For each of the claims, each dependent claim can depend both from the independent claim and from each of the prior dependent claims for each and every claim so long as the prior claim provides a proper antecedent basis for a claim term or element.

What is claimed is:

1. A method of treating a patient with a known or suspected of infection with a virus, viral-induced infection, respiratory disorder or exacerbation thereof, the method comprising: administering to the patient in need thereof a therapeutically effective amount of an agent, wherein the agent comprises at least one of:
a fusion protein comprising:
(a) an anti-viral-associated antibody or anti-epithelial-associated antibody (aVab/aEab)-IFN-Fc fusion protein, wherein the aVab/aEab is an anti-PD-L1, anti-VEGF, or anti-EGFR antibody variable domain;
(b) an activatable pro-IFN-Fc fusion protein (pro-IFN-Fc);
(c) a fusion protein comprising of an activatable pro-aVab/aEab-IFN-Fc, an activatable aVab/aEab-pro-IFN-Fc, or pro-aVab/aEab-pro-IFN-Fc; or
(d) an Fc-dimerized combination of the fusion proteins of (a)-(c).

2. The method of claim 1, wherein the virus is selected from the group consisting of Orthomyxoviridae, Paramyxoviridae, Picornaviridae, Rhabdoviridae, Coronaviridae, or Flaviviridae.

3. The method of claim 1, wherein the virus is seasonal influenza, a coronavirus, or SARS, SARS-CoV, MERS-CoV, 2019-nCoV virus, or nCoV strains of subsequent years.

4. The method of claim 1, wherein the pro-IFN is activated by a protease that is upregulated or secreted during a viral infection, or the protease is selected from membrane anchored MMPs MMP14 (MT1-MMP), MMP15 (MT2-MMP), MMP16 (MT3-MMP), MMP17 (MT4-MMP), MMP24 (MT5-MMP), MMP25 (MT5-MMP or leukolysin), or matrilysins MMP-7 and MMP-26, or stromelysins MMP3, MMP10, MMP11, MMP19, or gelatinases MMP2, MMP9, or collagenases MMP1, MMP8, MMP13, MMP18, or any one of caspase 1 to 9.

5. The method of claim 1, wherein the agent is formulated for administration by nasopharyngeal airway, oropharyngeal airway or intravenous delivery, or administered to the lung or a lower respiratory tract with an aerosol nebulizer.

6. The method of claim 1, wherein the agent has at least 90, 91, 92, 93, 94, 95, 96, 97, 98, 99 or 100% sequence identify with a fusion protein as set forth in SEQ ID NO:19 to 34, 38 and 39.

7. The method of claim 1, wherein the agent is administered simultaneously, separately or sequentially in combination with an additional therapeutic agent or an inhaled corticosteroid.

8. The method of claim 1, wherein the agent comprises a polynucleotide vector that expresses in a target bronchial epithelial cell the polynucleotide that expresses an activatable pro-IFN-Fc fusion antibody, an aVab/aEab-IFN-Fc fusion antibody, an activatable pro-aVab/aEab-IFN-Fc, an activatable aVab/aEab-pro-IFN-Fc, or pro-aVab/aEab-pro-IFN-Fc, wherein aVab/aEab is anti-VEGF, anti-EGFR or anti-PD-L1; and wherein administration of the agent results in suppression of viral replication causing a reduction in the viral-induced infection, respiratory disorder or exacerbation thereof in the patient.

9. The method of claim 1, wherein the IFN is selected from at least one of: IFN-α1, IFN-α2, IFN-α3, IFN-α4, IFN-α5, IFN-α6, IFN-α7, IFN-α8, IFN-α10, IFN-α13, IFN-α14, IFN-α16, IFN-α17, IFN-α21, IFN-β, IFN-ε, IFN-κ, IFN-ω.

10. The method of claim 1, wherein the pro-IFN is at least one of: pro-IFN-alpha, a pro-IFN-gamma, or a pro-IFN-lambda; the pro-IFN-alpha or pro-IFN-gamma further comprises an extracellular domain of IFNAR1 or IFNAR2; or the pro-IFN-lambda further comprises an extracellular domain of IFNLR1.

11. The method of claim 1, wherein the pro-IFN is defined further as a heterodimer selected from at least one of: anti-VEGF(scFv)-Fc6-IFN-Fc9, anti-VEGF(scFv)-Fc6-pro-IFN-Fc9, pro-anti-VEGF(scFv)-Fc6-IFN-Fc9, pro-anti-VEGF(scFv)-Fc6-pro-IFN-Fc9, anti-EGFR(scFv)-Fc6-IFN-Fc9, anti-EGFR(scFv)-Fc6-pro-IFN-Fc9, anti-PD-L1(scFv)-Fc6-IFN-Fc9, anti-PD-L1(scFv)-Fc6-pro-IFN-Fc9, pro-anti-PD-L1(scFv)-Fc6-IFN-Fc9, or pro-anti-PD-L1(scFv)-Fc6-pro-IFN-Fc9.

12. The method of claim 1, wherein the pro-IFN is defined further as a homodimer selected from at least one of: pro-IFN-Fc, anti-VEGF(scFv)-IFN-Fc, anti-VEGF(scFv)-pro-IFN-Fc, pro-anti-VEGF(scFv)-IFN-Fc, anti-EGFR(scFv)-IFN-Fc, anti-EGFR(scFv)-pro-IFN-Fc, anti-PD-L (scFv)-IFN-Fc, anti-PD-L (scFv)-pro-IFN-Fc, pro-anti-PD-L1(scFv)-IFN-Fc, or pro-anti-PD-L1(scFv)-pro-IFN-Fc, IFN or pro-IFN fusion constructs can be fused to an N-terminus or a C-terminus of Fc.

13. The method of claim 1, wherein the pro-IFN further comprises at least one of: a peptide, a receptor to IFN, or a portion of IFN receptor that binds to and reduces the activity of IFN, and is disassociated with IFN-Fc; the pro-IFN further comprises an Fc region; or the pro-IFN is activated in bronchi by proteases secreted by cells infected with a virus.

14. A method of administering to a patient prior to infection with a virus, viral-induced infection, respiratory disorder or exacerbation thereof, the method comprising:
administering to the patient in need thereof a prophylactically effective amount of a fusion protein comprising:
(a) aVab/aEab-IFN-Fc, wherein aVab/aEab is anti-PD-L1, anti-VEGF, or anti-EGFR antibody variable domain;
(b) an activatable pro-IFN-Fc fusion protein (pro-IFN-Fc) fusion protein comprising of an activatable pro-IFN (pro-IFN), X-pro-IFN or pro-IFN-X, wherein X is an anti-viral antibody;
(c) a fusion protein comprising of an activatable pro-aVab/aEab-IFN-Fc, an activatable aVab/aEab-pro-IFN-Fc, or pro-aVab/aEab-pro-IFN-Fc; or
(d) an Fc-dimerized combination of the fusion proteins of (a)-(c).

15. The method of claim 14, wherein the viral infection is a virus selected from the group consisting of Orthomyxoviridae, Paramyxoviridae, Picornaviridae, Rhabdoviridae, Coronaviridae, or Flaviviridae.

16. The method of claim 14, wherein the virus is seasonal influenza; or a coronavirus, or the virus is SARS, SARs-CoV, MERS-CoV, or 2019-nCoV virus.

17. The method of claim 14, wherein the fusion protein is a pro-IFN activated by a protease that is upregulated or secreted during a viral infection; or the protease is selected from membrane anchored MMPs MMP14 (MT1-MMP), MMP15 (MT2-MMP), MMP16 (MT3-MMP), MMP17 (MT4-MMP), MMP24 (MT5-MMP), MMP25 (MT5-MMP or leukolysin), or matrilysins MMP-7 and MMP-26, or stromelysins MMP3, MMP10, MMP11, MMP19, or gelatinases MMP2, MMP9, or collagenases MMP1, MMP8, MMP13, MMP18, or any one of caspase 1 to 9.

18. The method of claim 14, wherein the fusion protein is formulated for administration by airway or intravenous delivery; or the fusion protein is administered to the lung or the lower respiratory tract with an aerosol nebulizer.

19. The method of claim 14, wherein the fusion protein has at least 90, 91, 92, 93, 94, 95, 96, 97, 98, 99 or 100% sequence identify with a fusion protein as set forth in SEQ ID NO:19 to 34, 38 and 39.

20. The method of claim 14, wherein the fusion protein is administered simultaneously, separately or sequentially in combination with an additional therapeutic agent; or the additional therapeutic agent is an inhaled corticosteroid.

21. The method of claim 14, wherein the fusion protein comprises a polynucleotide vector that expresses in a target bronchial epithelial cell the polynucleotide that expresses an activatable pro-IFN-Fc fusion antibody, an aVab/aEab-IFN-Fc fusion antibody, an activatable pro-aVab/aEab-IFN-Fc, an activatable aVab/aEab-pro-IFN-Fc, or pro-aVab/aEab-pro-IFN-Fc, wherein aVab/aEab is anti-VEGF, anti-EGFR or anti-PD-L1; and wherein administration of the fusion protein results in suppression of viral replication causing a reduction in the viral-induced infection, respiratory disorder or exacerbation thereof in the patient.

22. The method of claim 14, wherein the IFN is selected from at least one of: IFN-α1, IFN-α2, IFN-α3, IFN-α4, IFN-α5, IFN-α6, IFN-α7, IFN-α8, IFN-α10, IFN-α13, IFN-α14, IFN-α16, IFN-α17, IFN-α21, IFN-β, IFN-ε, IFN-κ, IFN-ω.

23. The method of claim 14, wherein the pro-IFN is at least one of: pro-IFN-alpha, a pro-IFN-gamma, or a pro-IFN-lambda; the pro-IFN-alpha or pro-IFN-gamma further comprises an extracellular domain of IFNAR1 or IFNAR2; or the pro-IFN-lambda further comprises an extracellular domain of IFNLR1.

24. The method of claim 14, wherein the pro-IFN is defined further as a heterodimer selected from at least one of: anti-VEGF(scFv)-Fc6-IFN-Fc9, anti-VEGF(scFv)-Fc6-pro-IFN-Fc9, pro-anti-VEGF(scFv)-Fc6-IFN-Fc9, pro-anti-VEGF(scFv)-Fc6-pro-IFN-Fc9, anti-EGFR(scFv)-Fc6-IFN-Fc9, anti-EGFR(scFv)-Fc6-pro-IFN-Fc9, anti-PD-L1(scFv)-Fc6-IFN-Fc9, anti-PD-L1(scFv)-Fc6-pro-IFN-Fc9, pro-anti-PD-L1(scFv)-Fc6-IFN-Fc9, or pro-anti-PD-L1(scFv)-Fc6-pro-IFN-Fc9.

25. The method of claim 14, wherein the pro-IFN is defined further as a homodimer selected from at least one of: pro-IFN-Fc, anti-VEGF(scFv)-IFN-Fc, anti-VEGF(scFv)-pro-IFN-Fc, pro-anti-VEGF(scFv)-IFN-Fc, anti-EGFR(scFv)-IFN-Fc, anti-EGFR(scFv)-pro-IFN-Fc, anti-PD-L1(scFv)-IFN-Fc, anti-PD-L1(scFv)-pro-IFN-Fc, pro-anti-PD-L1(scFv)-IFN-Fc, or pro-anti-PD-L1(scFv)-pro-IFN-Fc, IFN or pro-IFN fusion constructs can be fused to an N-terminus or a C-terminus of Fc.

26. The method of claim 14, wherein the pro-IFN further comprise at least one of: peptide, a receptor to IFN, or a portion of IFN receptor that binds to and reduces the activity of IFN, and is disassociated with IFN-Fc; the pro-IFN further comprises an Fc region; or the pro-IFN is activated in bronchi by proteases secreted by cells infected with a virus.

27. A fusion protein that comprises at least one of:
(a) aVab/aEab-IFN-Fc fusion protein, wherein aVab/aEab is anti-PD-L1, anti-VEGF, or anti-EGFR antibody variable domain;
(b) an activatable pro-IFN-Fc fusion protein (pro-IFN-Fc);
(c) a fusion protein comprising of an activatable pro-aVab/aEab-IFN-Fc, an activatable aVab/aEab-pro-IFN-Fc, or pro-aVab/aEab-pro-IFN-Fc; or
(d) an Fc-dimerized combination of the fusion proteins of (a)-(c).

28. The fusion protein of claim 27, wherein the fusion protein is at least one of: IFN (pro-IFN), X-pro-IFN or pro-IFN-X and is provided in an amount greater than 1 mg/kg per dose; or the fusion protein is IFN (pro-IFN), X-pro-IFN or pro-IFN-X and is provided in an amount of 1, 2, 3, 4, 5, 6, 7, 8, 9, 10, 15, 20, 25, 30, 35, 40, 45, 50, 55, 60 mg/kg per dose.

29. The fusion protein of claim 27, wherein the fusion protein has at least 90, 91, 92, 93, 94, 95, 96, 97, 98, 99 or 100% sequence identify with a fusion protein as set forth in SEQ ID NO: 19 to 34, 38 and 39.

30. A polynucleotide that encodes at least one fusion protein of claim 27.

31. A vector that comprises the polynucleotide of claim 30.

32. A host cell that comprises the vector of claim 31.

33. A method of making a fusion protein comprising expressing a ribonucleic acid that encodes at least one fusion protein of claim 27 under conditions in which the fusion protein is translated.
